(19) United States
(12) Patent Application Publication
(10) Pub. No.: US 2020/0283301 A1
(43) Pub. Date: Sep. 10, 2020

(54) HIGH ABSORPTION MINERALS

(71) Applicant: ImerTech SAS, Paris (FR)

(72) Inventors: Andrew Mark Riley, St Columb Major Cornwall (GB); Laure Pagis, Toulouse (FR); Anabelle Huguette Renée Elton-Legrix, St. Austell Cornwall (GB); Bo Wang, Union City, CA (US)

(73) Assignee: ImerTech SAS

(21) Appl. No.: 16/884,161

(22) Filed: May 27, 2020

Related U.S. Application Data
(63) Continuation of application No. 15/774,676, filed on May 9, 2018.

(30) Foreign Application Priority Data
Nov. 10, 2015 (EP) 15306790.5

(51) Int. Cl. C01B 33/24; B01J 20/10; B01J 20/14; B01J 20/30; B01J 20/28; C05G 5/00; A23K 20/28; A61K 8/25; A61Q 15/00; C01B 33/18

(52) U.S. Cl. CPC C01B 33/24; C01P 2004/34; B01J 20/14; B01J 20/106; B01J 20/3028; B01J 20/2803; B01J 20/28011; C05G 5/00; A23K 20/28; A61K 8/25; A61Q 15/00; B01J 20/103; B01J 20/28021; B01J 20/3042; C01B 33/18; B01J 20/10

(57) ABSTRACT

Minerals (e.g. silica- or silicate-based minerals) having good oil- and/or water-absorption capacities and/or good flowability, mineral composites comprising first and second mineral components having good oil- and/or water-absorption capacities and/or good flowability, methods of making said minerals and mineral composites and the uses of said minerals and mineral composites, for example in animal feed or fertilizer compositions.

### TECHNICAL HELD

The present invention generally relates to minerals and mineral composites having high acid and/or oil and/or water absorption capacities. The present invention further relates to methods for preparing these minerals and mineral composites and the various uses of these minerals and mineral composites. The present invention also relates to functional compositions and/or products (e.g. personal care products such as deodorant, animal feed compositions, fertilizer compositions) comprising these minerals and mineral composites.

### BACKGROUND OF THE INVENTION

Mineral materials can be used for a wide range of applications in a wide range of products. One use of mineral materials is for absorption of substances such as water and/or organic compounds such as organic acids and oil in numerous applications. It is therefore desirable to provide alternative and/or advantageous minerals and mineral compositions/composites. These alternative and/or advantageous minerals may each be suitable for use in particular applications.

### SUMMARY OF THE INVENTION

In accordance with a first aspect of the present invention, there is provided a silica- or silicate-based mineral that may be suitable for and/or intended for use in absorbing organic compounds such as organic acids and oil, and/or water. In certain embodiments of any aspect of the invention, the mineral is derived from a natural mineral. In accordance with further aspects of the present invention, there is provided methods of making the minerals, the various uses of the minerals, and functional compositions comprising the minerals.

In certain embodiments of any aspect of the invention, the mineral has an organic acid absorption capacity and/or an oil absorption capacity equal to or greater than about 140 g/100 g of the mineral. In certain embodiments, the mineral is in the form of a free-flowing granulate. Thus, in accordance with a second aspect of the present invention, there provided a silica- or silicate-based mineral that:
a) has an organic acid absorption capacity and/or an oil absorption capacity equal to or greater than about 140 g/100 g of the mineral; and/or
b) is in the form of a free-flowing granulate. In certain embodiments, the mineral is derived from a natural mineral.

In certain embodiments of any aspect of the invention, the mineral may be spray-dried. Thus, in accordance with a third aspect of the present invention, there is provided a method of making a mineral granulate, the method comprising:
spray-drying a suspension comprising particles of a silica-based mineral or silicate-based mineral, a liquid medium and a binder; and
recovering a spray dried mineral granulate. In certain embodiments, the silica-based mineral or silicate-based mineral is in accordance with any aspect or embodiment of the invention described herein.

In certain embodiments of any aspect of the invention, the recovered spray-dried mineral has an organic acid absorption capacity and/or an oil absorption capacity equal to or greater than about 140 g/100 g of the mineral and/or is in the form of a free-flowing granulate.

In certain embodiments of any aspect of the invention, a spray-dried mineral has a higher organic acid and/or oil and/or water absorption capacity than the mineral prior to spray-drying. Thus, in accordance with a fourth aspect of the present invention, there is provided a method for increasing the organic acid and/or oil and/or water absorption capacity of a mineral, comprising:
spray-drying a suspension comprising particles of the mineral, a liquid medium and a binder; and
recovering a spray dried mineral granulate that has an increased organic acid and/or oil and/or water absorption capacity in comparison to the respective organic acid and/or oil and/or water absorption capacity of the mineral prior to the spray-drying step. In certain embodiments, the mineral is a silica-based mineral or silicate-based mineral, for example in accordance with any aspect or embodiments of the invention described herein.

In accordance with a fifth aspect of the present invention, there is provided a mineral obtained by or obtainable by the method of the third or fourth aspect of the present invention.

In accordance with a sixth aspect of the present invention, there is provided a spray-dried silica-based or silicate-based mineral that:
a) has an organic acid absorption capacity and/or oil absorption capacity equal to or greater than about 140 g/100 g of the mineral; and/or
b) is in the form of a free-flowing granulate; and/or
c) has an organic acid and/or oil and/or water absorption capacity that is greater than the respective organic acid and/or oil and/or water absorption capacity of the mineral prior to spray-drying.

In certain embodiments of any aspect of the present invention, the mineral is perlite, diatomaceous earth and/or calcium silicate.

In accordance with further aspects of the present invention, there is provided a combination of two or more silica- or silicate-based minerals that may be suitable for and/or intended for use in absorbing organic acid and/or oil and/or water. Each of the silica- or silicate-based mineral components may be in accordance with any one or more of the aspects or embodiments described above. The overall organic acid and/or oil and/or water absorption capacity of the composition may, for example, be greater than the respective mean organic acid and/or oil and/or water absorption capacity of the mineral components.

Thus, in accordance with a seventh aspect of the present invention, there is provided a composite comprising a first silica- or silicate-based mineral and a second silica- or silicate-based mineral that is different to the first silica- or silicate-based mineral, wherein the composite:
a) has an organic acid absorption capacity equal to or greater than about 140 g/100 g of the composite; and/or
b) has an oil absorption capacity equal to or greater than about 140 g/100 g of the composite; and/or
c) has a water absorption capacity equal to or greater than about 140 g/100 g of the composite; and/or
d) has an organic acid and/or oil and/or water absorption capacity that is greater than the respective mean organic acid and/or oil and/or water absorption capacity of the silica- or silicate-based mineral components.

In certain embodiments of any aspect of the present invention, the first and/or second silica- or silicate-based mineral(s) are selected from perlite, diatomaceous earth and calcium silicate. In certain embodiments, the first silica- or silicate-based mineral is perlite and the second silica- or silicate-based mineral is calcium silicate (e.g. calcium silicate derived from a natural mineral).

In accordance with a sixth aspect of the present invention, there is provided a method for making a composite (e.g. the composite of the seventh aspect) comprising combining two or more silica- or silicate-based minerals.

In accordance with further aspects of the present invention, there is provided the various uses of the minerals and mineral compositions and composites disclosed herein. In one aspect, there is provided the use of the minerals and mineral compositions and composites according to any aspect or embodiment of the present invention to absorb a substance, such as water and/or oil and/or organic acid, or to absorb a water-based substance or organic acid-based substance or oil-based substance.

In accordance with an eighth aspect of the present invention, there is provided the use of a mineral or mineral composite according to any aspect or embodiment of the present invention as a carrier in a functional composition. In certain embodiments, the functional composition is an animal feed composition or a fertilizer composition.

In accordance with a ninth aspect of the present invention, there is provided the use of a mineral or mineral composite according to any aspect or embodiment of the present invention in a personal care product.

In accordance with further aspects of the present invention, there is provided functional compositions comprising the minerals or mineral composites according to any aspect of embodiment of the present invention. In certain embodiments, the functional composition is an animal feed composition, a fertilizer composition or a personal care product.

Certain embodiments of any aspect of the present invention, may provide one or more of the following advantages:
good (for example, high or increased) organic acid absorption properties;
good (for example, high or increased) oil absorption properties;
good (for example, high or increased) water absorption properties;
good (for example, high or increased) odour absorption;
good flowability (for example, even at relatively high oil and/or water absorption);
reduced use of synthetic materials;
improvement of (for example quick or quicker) drying time when used in liquid formulations;
improvement of texture when used in liquid formulations and applied to skin.

The details, examples and preferences provided in relation to any particular one or more of the stated aspects of the present invention will be further described herein and apply equally to all aspects of the present invention. Any combination of the embodiments, examples and preferences described herein in all possible variations thereof is encompassed by the present invention unless otherwise indicated herein, or otherwise dearly contradicted by context.

### BRIEF DESCRIPTION OF THE FIGURES

FIG. 1 shows the base flow energy of various minerals at different liquid (water or organic acid) loading levels.

### DETAILED DESCRIPTION OF THE INVENTION

### Minerals

There is provided herein minerals that are suitable for and/or intended for absorbing substances such as oil and/or water.

In certain embodiments, the mineral is a silica-based mineral or a silicate-based mineral. A silica-based mineral is a mineral that comprises greater than 50 wt % silica (SiO₂). A silicate-based mineral is a mineral that comprises greater than 50 wt % of a chemical compound that comprises silicate ions (e.g. orthosilicate ions (SiO₄⁴⁻) or other silicate ions ([SiO₂₊ₙ]²ⁿ⁻)). For example, a silica-based mineral may be a mineral that comprises greater than 60 wt % or greater than 70 wt % or greater than 80 wt % or greater than 90 wt % silica. For example a silicate-based mineral may be a mineral that comprises greater than 60 wt % or greater than 70 wt % or greater than 80 wt % or greater than 90 wt % of a chemical compound that comprises silicate ions.

In certain embodiments, the mineral is naturally-derived (derived from a natural mineral). In certain embodiments, the mineral is synthetic. “Naturally-derived” means that the mineral is either naturally occurring or is made from a naturally occurring mineral. Any mineral that is naturally occurring or made from a naturally occurring mineral is not synthetic. Where the mineral is naturally-derived, it may be that some mineral impurities will inevitably contaminate the ground material. In general, however, the mineral will contain less than 5% by weight, preferably less than 1% by weight of other mineral impurities.

Silica-based minerals include both naturally-derived silica-based minerals and synthetic silica-based minerals. Naturally-derived silica-based minerals include, for example, free silica, natural glasses, diatomaceous earth or mixtures thereof.

Free silica includes, for example, quartz, tridymite, cristobalite, opal, vitreous silica, coesite, stishovite and chalcedony.

Natural glasses (commonly referred to as volcanic glasses) are formed by the rapid cooling of siliceous magma or lava. Several types of natural glasses are known, including, for example, perlite, pumice, pumicite, shirasu, obsidian, and pitchstone. Prior to processing, perlite may be gray to green in color with abundant spherical cracks that cause it to break into small pearl-like masses. Pumice is a lightweight glassy vesicular rock. Obsidian may be dark in color with a vitreous luster and a characteristic conchoidal fracture. Pitchstone has a waxy resinous luster and may be brown, green, or gray. Volcanic glasses such as perlite and pumice occur in massive deposits and find wide commercial use. Volcanic ash, often referred to as “tuff” when in consolidated form, includes small particles or fragments that may be in glassy form.

As used herein, the term natural glass encompasses volcanic ash. Natural glasses may be chemically equivalent to rhyolite. Natural glasses that are chemically equivalent to trachyte, dacite, andesite, latite, and basalt are known but may be less common. The term “obsidian” is generally applied to large numbers of natural glasses that are rich in silica. Obsidian glasses may be classified into subcategories according to their silica content, with rhyolitic obsidians (containing typically about 73% SiO₂ by weight) being the most common.

Perlite is a hydrated natural glass that may contain, for example, about 72 to about 75% SiO₂, about 12 to about 14% Al₂O₃, about 0.5 to about 2% Fe₂O₃, about 3 to about 5% Na₂O, about 4 to about 5% K₂O, about 0.4 to about 1.5% CaO (by weight), and small amounts of other metallic elements. Perlite may be distinguished from other natural glasses by a higher content (such as about 2 to about 5% by weight) of chemically-bonded water, the presence of a vitreous, pearly luster, and characteristic concentric or arcuate onion skin-like (i.e., perlitic) fractures. Perlite may be expanded or non-expanded. Perlite products may be prepared by milling and thermal expansion, and may possess unique physical properties such as high porosity, low bulk density, and chemical inertness. Average particle size for the milled expanded perlite ranges from 5 to 200 microns, pore volume ranges from 2 to 10 l/mg with median pore size from 5 to 20 microns.

Pumice is a natural glass characterized by a mesoporous structure (e.g. having pores or vesicles with a size up to about 1 mm). The porous nature of pumice gives it a very low apparent density, in many cases allowing it to float on the surface of water. Most commercial pumice contains from about 60% to about 70% SiO₂ by weight. Pumice may be processed by milling and classification, and products may be used as lightweight aggregates and also as abrasives, adsorbents, and fillers. Unexpanded pumice and thermally-expanded pumice may also be used as filtration components.

Diatomaceous earth products may be obtained from diatomaceous earth (also called “DE” or “diatomite” or “kieselgur”), which is generally known as a sediment-enriched in biogenic silica (i.e., silica produced or brought about by living organisms) in the form of siliceous skeletons (frustules) of diatoms. Diatoms are a diverse array of microscopic, single-celled, golden-brown algae or algae-like plants generally of the class Bacillariophyceae that possess an ornate siliceous skeleton of varied and intricate structures including two valves that, in the living diatom, fit together much like a pill box. Diatomaceous earth may form from the remains of water-borne diatoms and, therefore, diatomaceous earth deposits may be found close to either current or former bodies of water. Those deposits are generally divided into two categories based on source: freshwater and saltwater. Freshwater diatomaceous earth is generally mined from dry lakebeds and may be characterized as having a low crystalline silica content and a high iron content. In contrast, saltwater diatomaceous earth is generally extracted from oceanic areas and may be characterized as having a high crystalline silica content and a low iron content.

Diatomaceous earth is principally composed of the silica microfossils of aquatic unicellular algae known as diatoms. Diatomaceous earth typically has a chemical composition in the range of about 60 to 95% silica, 1 to 12% alumina and 0.5 to 8% iron oxide. It may also contain small amounts of other compounds such as calcium oxide, titanium dioxide, magnesium oxide, sodium oxide and potassium oxide. Diatomaceous earth has a highly porous structure, for example containing up to 80 to 90% voids, and consists of particles of a wide variety of shapes and sizes. In one embodiment, natural diatomaceous earth comprises about 90% SiO₂ mixed with other substances. In another embodiment, crude diatomaceous earth comprises about 90% SiO₂, plus various metal oxides, such as but not limited to Al, Fe, Ca, and Mg oxides.

The diatomaceous earth starting material may have any of various appropriate forms now known to the skilled artisan or hereafter discovered. In one embodiment, the at least one natural diatomaceous earth is unprocessed (e.g., not subjected to chemical and/or physical modification processes). Without wishing to be bound by theory, the impurities in natural diatomaceous earth, such as clays and organic matters, may, in some embodiments, provide higher cation exchange capacity. The diatomaceous earth may, for example, have an average particle size ranging from about 5 to about 200 microns. The diatomaceous earth may, for example, have a surface area ranging from 1 to 80 m²/g. The diatomaceous earth may, for example have a pore volume ranging from 2 to 10 L/mg with a media pore size from 1 to 20 microns.

Where the mineral is diatomaceous earth, the mineral may have a low cristobalite content. For example, the cristobalite content may be less than about 2% by weight. For example, the cristobalite content may be less than about 1% by weight. For example, the cristobalite content may be less than about 0.5% by weight. For example, the cristobalite content may be less than about 0.1% by weight. Cristobalite content may be measured by any appropriate measurement technique now known to the skilled artisan or hereafter discovered, including the specific method described in WO 2010/042614.

Where the mineral is diatomaceous earth, the mineral may comprise at least one soluble metal. As used herein, the term “soluble metal” refers to any metal that may be dissolved in at least one liquid. Soluble metals are known to those of skill in the art and include, but are not limited to, iron, aluminium, calcium, vanadium, chromium, copper, zinc, nickel, cadmium, and mercury. When a filter aid comprising diatomaceous earth is used to filter at least one liquid, at least one soluble metal may dissociate from the diatomaceous earth filter aid and enter the liquid. Any appropriate protocol or test for measuring levels of at least one soluble metal in diatomaceous earth products may be used, including those now known to the skilled artisan or hereafter discovered. For example, the brewing industry has developed at least one protocol to measure the BSI of diatomaceous earth filter aids. BSI, or beer soluble iron, refers to the iron content, which may be measured in parts per million, of a filter aid comprising diatomaceous earth that dissociates in the presence of a liquid, such as beer. The European Beverage Convention (EBC) method contacts liquid potassium hydrogen phthalate with the filter aid and then analyzes the liquid for iron content. More specifically, the EBC method uses, for example, a 10 g/L solution of potassium hydrogen phthalate (KHP, KHC₈H₄O₄) as the extractant along with a given quantity of filter aid material, with a total contact time of two hours. Extracts are then analyzed for iron concentration by the FERROZINE method.

Where the mineral is diatomaceous earth, the mineral may have a permeability suitable for use in a filter aid composition. Permeability may be measured by any appropriate measurement technique now known to the skilled artisan or hereafter discovered. Permeability is generally measured in darcy units or darcy, as determined by the permeability of a porous bed 1 cm high and with a 1 cm² section through which flows a fluid with a viscosity of 1 mPa·s with a flow rate of 1 cm′/sec under an applied pressure differential of 1 atmosphere. The principles for measuring permeability have been previously derived for porous media from Darcy's law (see, for example, J. Bear, “The Equation of Motion of a Homogeneous Fluid: Derivations of Darcy's Law,” in Dynamics of Fluids in Porous Media 161-177 (2nd ed. 1988)). An array of devices and methods are in existence that may correlate with permeability. In one exemplary method useful for measuring permeability, a specially constructed device is designed to form a filter cake on a septum from a suspension of filtration media in water; the time required for a specified volume of water to flow through a measured thickness of filter cake of known cross-sectional area is measured. Thus, in an embodiment, the product described herein may have a permeability of at least 1.0 Da, preferably at least 3.0 Da.

Synthetic silica-based minerals include, for example, fumed silica, silica fume, precipitated silica and silica gel. In certain embodiments, the silica-based mineral is not a synthetic silica.

Silicate-based minerals include both naturally-derived silicate-based minerals and synthetic silicate-based minerals. Naturally-derived silicate-based minerals include, for example, orthosilicates (e.g. andalusite, mullite), sorosilicates, cyclosilicates (e.g. bentonite, tourmaline), inosilicate (e.g. ferrolsilite, wollastonite), phyllosilicates (e.g. chrystolite, clays such as halloysite, kaolinite, montmorillonite, vermiculite, talc and pyrophyllite, mica) and tectosilicates (e.g. feldspar). The silicate-based mineral may, for example, be any one or more of these silicates. For example, the silicate-based mineral may be calcium silicate, magnesium silicate or combinations thereof.

Naturally-derived silicate-based minerals also include minerals that are made from a naturally occurring mineral. For example, calcium silicate may be made by reacting calcium oxide with a naturally-occurring silica- or silicate-based mineral. For example, calcium silicate may be made by reacting calcium oxide with diatomaceous earth.

Calcium silicate that is made by reacting calcium oxide with diatomaceous earth may, for example, have a diatom-type mineral structure. The calcium silicate may, for example, have a pore volume equal to or greater than about 5.5 mL/g, for example equal to or greater than about 6.0 mL/g.

In one embodiment, the mineral undergoes minimal processing following mining or extraction. In a further embodiment, the mineral is subjected to at least one physical modification process. The skilled artisan will readily know physical modification processes appropriate for use, which may be now known or hereafter discovered; appropriate physical modification processes include, but are not limited to, milling, drying, and air classifying. In yet another embodiment, the mineral is subjected to at least one chemical modification process. The skilled artisan will readily know chemical modification processes appropriate for use in the present inventions, which may be now known or hereafter discovered; appropriate chemical modification processes include but are not limited to, silanization and calcination.

The present invention may tend to be discussed in terms of naturally-derived silica- or silicate-based minerals. For example, the present invention may tend to be discussed in terms of natural glass (e.g. perlite), diatomaceous earth or naturally-derived calcium silicate. For example, in certain embodiments, the mineral is diatomaceous earth. For example, in certain embodiments, the mineral is perlite. In certain embodiments, the perlite may be expanded and un-milled perlite. The expanded and un-milled perlite may, for example, have a d₅₀ ranging from about 250 to about 450 μm, for example from about 300 to about 400 μm. In certain embodiments, the perlite may be expanded and classified (e.g. roughly classified) perlite. The expanded and classified (e.g. roughly classified) perlite may, for example, have a d₅₀ ranging from about 45 to about 75 μm, for example from about 50 to about 70 μm. In certain embodiments, the perlite may be expanded and milled perlite. The expanded and milled perlite may, for example, have a d₅₀ ranging from about 20 to about 60 μm, for example from about 30 to about 50 μm. However, the invention should not be construed as being limited to such embodiments.

The composite materials disclosed herein have a particle size. Particle size may be measured by any appropriate measurement technique now known to the skilled artisan or hereafter discovered. In one exemplary method, particle size and particle size properties, such as particle size distribution (“psd”), are measured using a Leeds and Northrup Microtrac X100 laser particle size analyzer (Leeds and Northrup, North Wales, Pa. USA), which can determine particle size distribution over a particle size range from 0.12 μm to 704 μm. The size of a given particle is expressed in terms of the diameter of a sphere of equivalent diameter that sediments through the suspension, also known as an equivalent spherical diameter or “esd.” The median particle size, or d₅₀ value, is the value at which 50% by weight of the particles have an esd less than that d₅₀ value. The d₁₀ value is the value at which 10% by weight of the particles have an esd less than that d₁₀ value. The d₉₀ value is the value at which 90% by weight of the particles have an esd less than that d₉₀ value.

The silica- or silicate-based mineral may, for example, have a d₁₀ ranging from about 0.5 to about 150 μm. For example, the mineral may have a d₁₀ ranging from about 0.5 to about 130 μm, for example from about 0.5 to about 120 μm, for example from about 0.5 to about 100 μm, for example from about 0.5 to about 50 μm, for example from about 0.5 to about 30 μm, for example from about 0.5 to about 20 μm, for example from about 0.5 to about 10 μm, for example from about 0.5 to about 5 μm. For example, the mineral may have a d₁₀ ranging from about 1 to about 10 μm, for example from about 2 to about 10 μm, for example from about 2 to about 20 μm. For example, the mineral may have a d₁₀ ranging from about 80 to about 150 μm, for example from about 90 to about 140 μm, for example from about 90 to about 130 μm, for example from about 100 to about 120 μm.

The silica- or silicate-based mineral may, for example, have a d₅₀ ranging from about 5 to about 450 μm. For example, the mineral may have a d₅₀ ranging from about 5 to about 100 μm, for example from about 5 to about 80, for example from about 5 to about 60 μm, for example from about 5 to about 50 μm, for example from about 5 to about 40 μm, for example from about 5 to about 30 μm, for example from about 5 to about 20 μm. For example, the mineral may have a d₅₀ ranging from about 20 to about 180 μm, for example from about 30 to about 170 μm, for example from about 40 to about 160 μm. For example, the mineral may have a d₅₀ ranging from about 200 to about 450 μm, for example from about 250 to about 450 μm, for example from about 300 to about 450 μm or from about 250 to about 400 μm.

The silica- or silicate based mineral may, for example, have a d₉₀ ranging from about 10 to about 1000 μm. For example, the mineral may have a d₉₀ ranging from about 15 to about 120 μm, for example from about 15 to about 100 μm, for example from about 15 to about 90 μm, for example from about 15 to about 80 μm, for example from about 15 to about 70 μm, for example from about 15 to about 60 μm, for example from about 15 to about 60 μm, for example from about 15 to about 50 μm, for example from about 15 to about 40 μm, for example from about 15 to about 30 μm, for example from about 20 to about 30 pin. For example, the mineral may have a d₉₀ ranging from about 40 to about 100 μm, for example from about 50 to about 90 μm, for example from about 50 to about 80 μm. For example, the mineral may have a d₉₀ ranging from about 100 to about 300 μm, for example from about 100 to about 200 μm, for example from about 100 to about 150 μm. For example, the mineral may have a d₉₀ ranging from about 600 to about 1000 μm, for example from about 600 to about 800 μm or from about 800 to about 1000 μm, for example from about 700 to about 900 μm.

In certain embodiments, the mineral is diatomaceous earth having a d₁₀ ranging from about 0.5 to about 150 μm. For example, the mineral may be diatomaceous earth having a d₁₀ ranging from about 0.5 to about 30 μm, for example from about 0.5 to about 20 μm, for example from about 0.5 to about 10 μm, for example from about 0.5 to about 5 μm. For example, the mineral may be spray-dried diatomaceous earth having a d₁₀ ranging from about 5 to about 150 μm, for example from about 5 to about 140 μm, for example from about 5 to about 130 μm, for example from about 5 to about 120 μm. For example, the mineral may be spray-dried diatomaceous earth having a d₁₀ ranging from about 5 to about 30 μm, for example from about 5 to about 25 μm, for example from about 5 to about 20 μm, for example from about 5 to about 15 μm. For example, the mineral may be granulated diatomaceous earth having a d₁₀ ranging from about 0.5 to about 10 μm, for example from about 0.5 to about 8 μm, for example from about 0.5 to about 6 μm, for example form about 0.5 to about 5 μm.

In certain embodiments, the mineral is diatomaceous earth having a d₅₀ ranging from about 5 to about 200 μm. For example, the mineral may be diatomaceous earth having a d₅₀ ranging from about 5 to about 60 μm, for example from about 5 to about 50 μm, for example from about 5 to about 40 μm, for example from about 5 to about 30 μm, for example from about 5 to about 20 μm, for example from about 5 to about 15 μm. For example, the mineral may be spray-dried diatomaceous earth having a d₅₀ ranging from about 20 to about 180 μm, for example from about 20 to about 170 μm, for example from about 20 to about 160 μm, for example from about 100 to about 180 μm. For example, the mineral may be spray-dried diatomaceous earth having a d₅₀ ranging from about 20 to about 60 μm, for example from about 20 to about 50 μm, for example from about 20 to about 40 μm, for example from about 30 to about 50 μm. For example, the mineral may be granulated diatomaceous earth having a d₅₀ ranging from about 10 to about 40 μm, for example from about 10 to about 30 μm, for example from about 15 about 30 μm, for example form about 15 to about 25 μm.

In certain embodiments, the mineral is diatomaceous earth having a d₉₀ ranging from about 10 to about 300 μm. For example, the mineral may be diatomaceous earth having a d₅₀ ranging from about 15 to about 270 μm, for example from about 15 to about 260 μm, for example from about 15 to about 250 μm, for example from about 15 to about 100 μm, for example from about 15 to about 80 μm, for example from about 15 to about 60 μm, for example from about 15 to about 50 μm, for example from about 15 to about 40 μm, for example from about 15 to about 30 μm. For example, the mineral may be spray-dried diatomaceous earth having a d₅₀ ranging from about 50 to about 300 μm, for example from about 50 to about 280 μm, for example from about 50 to about 260 μm, for example from about 50 to about 250 μm. For example, the mineral may be spray-dried diatomaceous earth having a d₅₀ ranging from about 50 to about 100 μm, for example from about 50 to about 90 μm, for example from about 50 to about 80 μm, for example from about 50 to about 70 μm. For example, the mineral may be granulated diatomaceous earth having a d₅₀ ranging from about 10 to about 50 μm, for example from about 20 to about 50 μm, for example from about 30 to about 50 μm, for example form about 30 to about 40 μm.

In certain embodiments, the mineral is perlite having a d₁₀ ranging from about 1 to about 150 μm. For example, in certain embodiments, the mineral is perlite having a d₁₀ ranging from about 5 to about 150 μm, for example from about 10 to about 150 μm, for example from about 10 to about 130 μm, for example from about 10 to about 120 μm, for example from about 10 to about 100 μm, for example form about 10 to about 50 μm, for example form about 10 to about 30 μm, for example from about 10 to about 20 μm. In certain embodiments, the mineral is expanded and un-milled perlite having a d₁₀ ranging from about 50 to about 150 μm, for example from about 60 to about 150 μm, for example from about 80 to about 150 μm, for example from about 90 to about 150 μm, for example from about 90 to about 150 μm. In certain embodiments, the mineral is expanded and un-milled perlite having a d₁₀ ranging from about 50 to about 140 μm, for example from about 60 to about 130 μm, for example from about 70 to about 120 μm, for example from about 80 to about 110 μm. In certain embodiments, the mineral is expanded and classified (e.g. roughly classified) perlite having a d₁₀ ranging from about 10 to about 50 μm, for example from about 15 to about 40 μm, for example from about 15 to about 30 μm, for example from about 15 to about 25 μm, for example from about 15 to about 20 μm. In certain embodiments, the mineral is expanded and milled perlite having a d₁₀ ranging from about 10 to about 30 μm, for example from about 10 to about 20 μm, for example from about 10 to about 15 μm.

In certain embodiments, the mineral is perlite having a d₅₀ ranging from about 10 to about 450 μm. For example, in certain embodiments, the mineral is perlite having a d₁₀ ranging from about 20 to about 450 μm, for example from about 30 to about 450 μm, for example from about 30 to about 420 μm, for example from about 30 to about 400 μm. In certain embodiments, the mineral is expanded and un-milled perlite having a d₅₀ ranging from about 200 to about 450 μm, for example from about 250 to about 450 μm, for example from about 300 to about 450 μm, for example from about 300 to about 400 μm, for example from about 300 to about 4000 μm. In certain embodiments, the mineral is expanded and classified (e.g. roughly classified) perlite having a d₅₀ ranging from about 30 to about 90 μm, for example from about 40 to about 80 μm, for example from about 50 to about 70 μm, for example from about 50 to about 60 μm. In certain embodiments, the mineral is expanded and milled perlite having a d₅₀ ranging from about 20 to about 60 μm, for example from about 30 to about 50 μm, for example from about 30 to about 40 μm.

In certain embodiments, the mineral is perlite having a d₉₀ ranging from about 20 to about 1000 μm. For example, in certain embodiments, the mineral is perlite having a d₉₀ ranging from about 40 to about 1000 μm, for example from about 100 to about 1000 μm, for example from about 600 to about 1000 μm. In certain embodiments, the mineral is expanded and un-milled perlite having a d₉₀ ranging from about 600 to about 1000 μm, for example from about 650 to about 950 μm, for example from about 700 to about 900 μm. In certain embodiments, the mineral is expanded and classified (e.g. roughly classified) perlite having a d₉₀ ranging from about 100 to about 160 μm, for example from about 120 to about 150 μm, for example from about 120 to about 140 μm, for example from about 125 to about 135 μm. In certain embodiments, the mineral is expanded and milled perlite having a d₉₀ ranging from about 20 to about 70 μm, for example from about 30 to about 60 μm, for example from about 40 to about 60 μm.

In certain embodiments, the mineral is calcium silicate having a d₁₀ ranging from about 0.5 to about 10 μm, for example ranging from about 1 to about 10 μm, for example ranging from about 2 to about 8 μm, for example ranging from about 5 to about 8 μm. In certain embodiments, the mineral is calcium silicate having a d₅₀ ranging from about 10 to about 30 μm, for example from about 15 to about 25 μm. In certain embodiments, the mineral is calcium silicate having a d₉₀ ranging from about 20 to about 50 μm, for example from about 30 to about 40 μm.

In certain embodiments, the mineral is able to absorb substances such as organic acid and/or oil and/or water. In certain embodiment, the mineral is able to absorb substances that are in suspension or solution with organic acid or oil or water.

In certain embodiments, the mineral has an organic acid absorption capacity equal to or greater than about 50 g/100 g of the mineral. For example, the mineral may have an organic acid absorption capacity equal to or greater than about 100 g/100 g of the mineral, for example equal to or greater than about 120 g/100 g of the mineral, for example equal to or greater than about 140 g/100 g of the mineral, for example equal to or greater than about 150 g/100 g of the mineral, for example equal to or greater than about 160 g/100 g of the mineral, for example equal to or greater than about 170 g/100 g of the mineral, for example equal to or greater than about 180 g/100 g of the mineral, for example equal to or greater than about 190 g/100 g of the mineral, for example equal to or greater than about 200 g/100 g of the mineral, for example equal to or greater than about 210 g/100 g of the mineral, for example equal to or greater than about 220 g/100 g of the mineral, for example equal to or greater than about 230 g/100 g of the mineral, for example equal to or greater than about 240 g/100 g of the mineral, for example equal to or greater than about 250 g/100 g of the mineral, for example equal to or greater than about 260 g/100 g of the mineral, for example equal to or greater than about 270 g/100 g of the mineral, for example equal to or greater than about 280 g/100 g of the mineral, for example equal to or greater than about 290 g/100 g of the mineral, for example equal to or greater than about 300 g/100 g of the mineral. For example, the mineral may have an organic acid absorption capacity equal to or greater than about 310 g/100 g of the mineral, for example equal to or greater than about 320 g/100 g of the mineral, for example equal to or greater than about 330 g/100 g of the mineral, for example equal to or greater than about 340 g/100 g of the mineral, for example equal to or greater than about 350 g/100 g of the mineral. For example, the mineral may have an organic acid absorption capacity equal to or greater than about 400 g/100 g of the mineral, for example equal to or greater than about 450 g/100 g of the mineral, for example equal to or greater than about 500 g/100 g of the mineral.

In certain embodiments, the mineral has an oil-absorption capacity equal to or greater than about 50 g/100 g of the mineral. For example, the mineral may have an oil-absorption capacity equal to or greater than about 100 g/100 g of the mineral, for example equal to or greater than about 120 g/100 g of the mineral, for example equal to or greater than about 140 g/100 g of the mineral, for example equal to or greater than about 150 g/100 g of the mineral, for example equal to or greater than about 160 g/100 g of the mineral, for example equal to or greater than about 170 g/100 g of the mineral, for example equal to or greater than about 180 g/100 g of the mineral, for example equal to or greater than about 190 g/100 g of the mineral, for example equal to or greater than about 200 g/100 g of the mineral, for example equal to or greater than about 210 g/100 g of the mineral, for example equal to or greater than about 220 g/100 g of the mineral, for example equal to or greater than about 230 g/100 g of the mineral, for example equal to or greater than about 240 g/100 g of the mineral, for example equal to or greater than about 250 g/100 g of the mineral, for example equal to or greater than about 260 g/100 g of the mineral, for example equal to or greater than about 270 g/100 g of the mineral, for example equal to or greater than about 280 g/100 g of the mineral, for example equal to or greater than about 290 g/100 g of the mineral, for example equal to or greater than about 300 g/100 g of the mineral. For example, the mineral may have an oil-absorption capacity equal to or greater than about 310 g/100 g of the mineral, for example equal to or greater than about 320 g/100 g of the mineral, for example equal to or greater than about 330 g/100 g of the mineral, for example equal to or greater than about 340 g/100 g of the mineral, for example equal to or greater than about 350 g/100 g of the mineral. For example, the mineral may have an oil-absorption capacity equal to or greater than about 400 g/100 g of the mineral, for example equal to or greater than about 450 g/100 g of the mineral, for example equal to or greater than about 500 g/100 g of the mineral.

In certain embodiments, the mineral may have an oil-absorption capacity ranging from about 50 to about 800 g/100 g of the mineral, for example from about 100 to about 800 g/100 g of the mineral, for example from about 200 to about 800 g/100 g of the mineral. For example, the mineral may have an oil-absorption capacity ranging from about 220 to about 800 g/100 g of the mineral, for example from about 220 to about 600 g/100 g of the mineral, for example from about 270 to about 800 g/100 g of the mineral, for example from about 270 to about 600 g/100 g of the mineral, for example from about 300 to about 800 g/100 g of the mineral, for example from about 300 to about 600 g/100 g of the mineral, for example from about 300 to about 500 g/100 g of the mineral. The present invention may tend to be discussed in terms of minerals having an oil-absorption capacity equal to or greater than about 200 g/100 g of the mineral. However, the invention should not be construed as being limited to such embodiments.

The oil-absorption capacity and organic acid absorption capacity may vary between different minerals. For example, natural glasses such as perlite may have an oil-absorption capacity equal to or greater than about 270 g/100 g of the mineral. For example, natural glasses such as perlite may have an oil-absorption capacity equal to or greater than about 280 g/100 g of the mineral, for example equal to or greater than about 290 g/100 g of the mineral, for example equal to or greater than about 300 g/100 g of the mineral. For example, natural glasses such as perlite may have an oil-absorption capacity ranging from about 270 to about 800 g/100 g of the mineral, for example from about 270 to about 500 g/100 g of the mineral, for example from about 270 to about 400 g/100 g of the mineral.

For example, natural glasses such as perlite may have an organic acid absorption capacity equal to or greater than about 200 g/100 g of mineral. For example, natural glasses such as perlite may have an organic acid absorption capacity equal to or greater than about 300 g/100 g of mineral, for example equal to or greater than about 320 g/100 g of mineral, for example equal to or greater than about 340 g/100 g of mineral, for example equal to or greater than about 400 g/100 g of mineral. For example, natural glasses such as expanded, un-milled perlite may have an organic acid absorption capacity equal to or greater than about 500 g/100 g of mineral, for example equal to or greater than about 550 g/100 g of mineral, for example equal to or greater than about 600 g/100 g of mineral, for example equal to or greater than about 650 g/100 g of mineral, for example equal to or greater than about 700 g/100 g of mineral.

Diatomaceous earth may, for example, have an oil-absorption capacity equal to or greater than about 220 g/100 g of the mineral. For example, diatomaceous earth may have an oil-absorption capacity equal to or greater than about 230 g/100 g of the mineral, for example equal to or greater than about 240 g/100 g of the mineral, for example equal to or greater than about 250 g/100 g of the mineral, for example equal to or greater than about 260 g/100 g of the mineral, for example equal to or greater than about 270 g/100 g of the mineral, for example equal to or greater than about 280 g/100 g of the mineral, for example equal to or greater than about 290 g/100 g of the mineral, for example equal to or greater than about 300 g/100 g of the mineral. For example, diatomaceous earth may have an oil-absorption capacity ranging from about 220 to about 600 g/100 g of the mineral, for example from about 220 to about 500 g/100 g of the mineral, for example from about 250 to about 400 g/100 g of the mineral, for example from about 300 to about 400 g/100 g of the mineral.

For example, diatomaceous earth may have an organic acid absorption capacity equal to or greater than about 200 g/100 g of mineral. For example, diatomaceous earth may have an organic acid absorption capacity equal to or greater than about 250 g/100 g of mineral, for example equal to or greater than about 300 g/100 g of mineral, for example equal to or greater than about 350/100 g of mineral, for example equal to or greater than about 350 g/100 g of mineral.

Calcium silicate (e.g. derived from a natural mineral (e.g. derived from diatomaceous earth)) may, for example, have an oil-absorption capacity equal to or greater than about 300 g/100 g of the mineral. For example, calcium silicate may have an oil-absorption capacity equal to or greater than about 310 g/100 g of the mineral, for example equal to or greater than about 320 g/100 g of the mineral, for example equal to or greater than about 330 g/100 g of the mineral, for example equal to or greater than about 340 g/100 g of the mineral, for example equal to or greater than about 350 g/100 g of the mineral. For example, calcium silicate may have an oil-absorption capacity ranging from about 300 to about 800 g/100 g of the mineral, for example from about 300 to about 600 g/100 g of the mineral.

In certain embodiments, the mineral has a water-absorption capacity equal to or greater than about 50 g/100 g of the mineral. For example, the mineral may have a water-absorption capacity equal to or greater than about 100 g/100 g of the mineral, for example equal to or greater than about 120 g/100 g of the mineral, for example equal to or greater than about 140 g/100 g of the mineral, for example equal to or greater than about 150 g/100 g of the mineral, for example equal to or greater than about 160 g/100 g of the mineral, for example equal to or greater than about 170 g/100 g of the mineral, for example equal to or greater than about 180 g/100 g of the mineral, for example equal to or greater than about 190 g/100 g of the mineral, for example equal to or greater than about 200 g/100 g of the mineral, for example equal to or greater than about 210 g/100 g of the mineral, for example equal to or greater than about 220 g/100 g of the mineral, for example equal to or greater than about 230 g/100 g of the mineral, for example equal to or greater than about 240 g/100 g of the mineral, for example equal to or greater than about 250 g/100 g of the mineral, for example equal to or greater than about 260 g/100 g of the mineral, for example equal to or greater than about 270 g/100 g of the mineral, for example equal to or greater than about 280 g/100 g of the mineral, for example equal to or greater than about 290 g/100 g of the mineral, for example equal to or greater than about 300 g/100 g of the mineral. For example, the mineral may have a water-absorption capacity equal to or greater than about 310 g/100 g of the mineral, for example equal to or greater than about 320 g/100 g of the mineral, for example equal to or greater than about 330 g/100 g of the mineral, for example equal to or greater than about 340 g/100 g of the mineral, for example equal to or greater than about 350 g/100 g of the mineral. For example, the mineral may have a water-absorption capacity equal to or greater than about 400 g/100 g of the mineral, for example equal to or greater than about 450 g/100 g of the mineral, for example equal to or greater than about 500 g/100 g of the mineral.

In certain embodiments, the mineral may have a water-absorption capacity ranging from about 50 to about 800 g/100 g of the mineral, for example from about 100 to about 800 g/100 g of the mineral, for example from about 200 to about 800 g/100 g of the mineral. For example, the mineral may have a water-absorption capacity ranging from about 220 to about 800 g/100 g of the mineral, for example from about 220 to about 600 g/100 g of the mineral, for example from about 270 to about 800 g/100 g of the mineral, for example from about 270 to about 600 g/100 g of the mineral, for example from about 300 to about 800 g/100 g of the mineral, for example from about 300 to about 600 g/100 g of the mineral, for example from about 300 to about 500 g/100 g of the mineral. The present invention may tend to be discussed in terms of minerals having a water-absorption capacity equal to or greater than about 200 g/100 g of the mineral. However, the invention should not be construed as being limited to such embodiments

For example, natural glasses such as perlite may have a water-absorption capacity equal to or greater than about 200 g/100 g of mineral. For example, natural glasses such as perlite may have a water-absorption capacity equal to or greater than about 300 g/100 g of mineral, for example equal to or greater than about 320 g/100 g of mineral, for example equal to or greater than about 340 g/100 g of mineral, for example equal to or greater than about 400 g/100 g of mineral. For example, natural glasses such as expanded, un-milled perlite may have a water-absorption capacity equal to or greater than about 500 g/100 g of mineral, for example equal to or greater than about 550 g/100 g of mineral, for example equal to or greater than about 600 g/100 g of mineral, for example equal to or greater than about 650 g/100 g of mineral; for example equal to or greater than about 700 g/100 g of mineral.

For example, diatomaceous earth may have a water-absorption capacity equal to or greater than about 200 g/100 g of mineral. For example, diatomaceous earth may have a water-absorption capacity equal to or greater than about 250 g/100 g of mineral, for example equal to or greater than about 300 g/100 g of mineral, for example equal to or greater than about 350 g/100 g of mineral, for example equal to or greater than about 350 g/100 g of mineral.

The organic acid absorption capacity, oil-absorption capacity and water-absorption capacities are determined by weighing a sample of mineral into a container (e.g. 1 to 10 grams into a 100 to 300 ml ceramic or glass dish) and adding either organic acid, oil or water to the mineral gradually in a dropwise manner (e.g. about 1 drop per second). The sample is stirred during the addition of the liquid so that each drop fails on a dry portion of the mineral sample. When the sample particles become wet with water, they coalesce and form small lumps of paste. These lumps should be kept distributed throughout the mass, using a minimum of stirring, and using care not to use pressure in the mixing. As the absorption of water progresses, the lumps of paste form larger lumps which, when stirred around, form balls. \Mien this point is reached, the rate and quantity of the water added should be decreased to ensure you do not go past the end point. When adding water at this point it should strike the balls, not the dry sample. These balls are stirred around to bring the watery surface into contact with the remaining dry sample. When the dry sample is wet and picked up, the paste lumps tend to smear on the sides and bottom of the casserole. This is the end point. The total amount of water used in noted and the g of liquid (organic acid/oil/water) per 100 g of mineral sample is calculated. The organic acid may, for example, be a blend of organic acids, for example a mixture of acetic acid and formic acid and propionic acid, for example the blend known as Fylax® available from Seiko. The oil may, for example, be linseed oil. The oil absorption of the samples may be determined on a weight basis according to ASTM-D1483-95. The oil absorption in weight percentage may be calculated as follows:

$$Oil Absorption ( wt . \% ) = \frac{Volume Oil Used ( mL ) \times Specific Gravity of Oil}{Weight of Sample ( g )} \times 100$$

In certain embodiments, the mineral is the form of a free-flowing granulate. “Free-flowing” means that the mineral can move freely, for example the mineral can move in a continuous stream (e.g. “poured”), “Granulate” means that the mineral is in the form of particles or grains (particles formed from more than one smaller particles).

Whether a mineral is in the form of a “free-flowing granulate” may be determined by its base flow energy (BFE). For example, a mineral may be considered to be a “free-flowing granulate” if it has a BFE equal to or less than about 1200 mJ. For example, a mineral may be considered to be “free-flowing” if it has a BFE equal to or less than about 1100 mJ, for example equal to or less than about 1000 mJ, for example equal to or less than about 800 mJ, for example equal to or less than about 700 mJ, for example equal to or less than about 600 mJ.

In certain embodiments, the mineral is in the form of a free-flowing granulate even at relatively high oil and/or water contents. For example, the mineral may be in the form of a free-flowing granulate at a liquid (e.g. organic acid and/or oil and/or water) content of at least about 140 g/100 g of the mineral. For example, the mineral may be in the form of a free-flowing granulate at a liquid content of at least about 150 g/100 g of the mineral, for example at least about 160 g/100 g of the mineral, for example at least about 170 g/100 g of the mineral, for example at least about 180 g/100 g of the mineral, for example at least about 190 g/100 g of the mineral, for example at least about 200 g/100 g of the mineral, for example at least about 210 g/100 g of the mineral, for example at least about 220 g/100 g of the mineral, for example at least about 230 g/100 g of the mineral, for example at least about 240 g/100 g of the mineral, for example at least about 250 g/100 g of the mineral, for example at least about 260 g/100 g of the mineral, for example at least about 270 g/100 g of the mineral, for example at least about 280 g/100 g of the mineral, for example at least about 290 g/100 g of the mineral, for example at least about 300 g/100 g of the mineral. For example, the mineral may be in the form of a free-flowing granulate at a liquid content ranging from about 140 to about 600 g/100 g of the mineral, for example from about 150 to about 550 g/100 g of the mineral, for example from about 160 to about 500 g/100 g of the mineral.

In certain embodiments, the mineral has a base flow energy (BFE) equal to or less that is equal to or less than about 1200 mJ when the mineral has a liquid content of 200 g/100 g of the mineral. For example, the mineral may have a BFE equal to or less than about 1100 mJ when the mineral has a liquid content of 200 g/100 g of the mineral, for example equal to or less than about 1000 mJ when the mineral has a liquid content of 200 g/100 g of the mineral, for example equal to or less than about 900 mJ when the mineral has a liquid content of 200 g/100 g of the mineral, for example equal to or less than about 800 mJ when the mineral has a liquid content of 200 g/100 g of the mineral. For example, the mineral may have a BFE ranging from about 200 to about 1200 mJ when the mineral has a liquid content of 200 g/100 g of the mineral, for example from about 300 to about 1100 mJ when the mineral has a liquid content of 200 g/100 g of the mineral, for example from about 400 to about 1000 mJ when the mineral has a liquid content of 200 g/100 g of the mineral, for example from about 400 to about 800 mJ when the mineral has a liquid content of 200 g/100 g of the mineral.

In certain embodiments, the mineral has a base flow energy (BFE) equal to or less that is equal to or less than about 1200 mJ when the mineral has an organic acid content of 185 g/100 g of the mineral. For example, the mineral may have a BFE equal to or less than about 1100 mJ when the mineral has an organic acid content of 185 g/100 g of the mineral, for example equal to or less than about 1000 mJ when the mineral has an organic acid content of 185 g/100 g of the mineral, for example equal to or less than about 900 mJ when the mineral has an organic acid content of 185 g/100 g of the mineral, for example equal to or less than about 800 mJ when the mineral has an organic acid content of 185 g/100 g of the mineral. For example, the mineral may have a BFE ranging from about 200 to about 1200 mJ when the mineral has an organic acid content of 185 g/100 g of the mineral, for example from about 300 to about 1100 mJ when the mineral has an organic acid content of 185 g/100 g of the mineral, for example from about 400 to about 1000 mJ when the mineral has an organic acid content of 185 g/100 g of the mineral, for example from about 400 to about 800 mJ when the mineral has an organic acid content of 185 g/100 g of the mineral.

In certain embodiments, the mineral has a base flow energy (BFE) that is equal to or less than about 1200 mJ when the mineral has a water content of 150 g/100 g of the mineral. For example, the mineral may have a BFE equal to or less than about 1100 mJ when the mineral has a water content of 150 g/100 g of the mineral, for example equal to or less than about 1000 mJ when the mineral has a water content of 150 g/100 g of the mineral, for example equal to or less than about 900 mJ when the mineral has a water content of 150 g/100 g of the mineral, for example equal to or less than about 800 mJ when the mineral has a water content of 150 g/100 g of the mineral. For example, the mineral may have a BFE ranging from about 200 to about 1200 mJ when the mineral has a water content of 150 g/100 g of the mineral, for example from about 300 to about 1100 mJ when the mineral has a water content of 150 g/100 g of the mineral, for example from about 400 to about 1000 mJ when the mineral has a water content of 150 g/100 g of the mineral, for example from about 400 to about 800 mJ when the mineral has a water content of 150 g/100 g of the mineral.

Base flow energy is measured using a Freeman Powder Rheometer model FT3. The FT3 rheometer drives a blade along a helical path downward through a powder sample. As the blade forces it way down through the powder the force imposed upon it is measured. It is this data that forms the basis of the measurements made. The helical path that the blade takes through the sample is determined by a combination of the rotational and axial speeds. Each particle within the powder mass lies at a state of rest until forced to move, coming to rest again as the blade moves on. The pattern of powder displacement is virtually steady state, allowing flow to be observed and generally resulting in smooth, linear or logarithmic profiles of the measured forces. These forces are those required to initiate shearing and breakdown of interparticulate bonding of the powder in the zone immediately around the blade, a process that is continuous.

The base flow energy is the energy required to displace a constant volume of conditioned powder at a given flow pattern and flow rate. Samples are prepared by measuring 160 ml of powder into the sample vessel and recording the mass. A conditioning cycle is then carried out on the sample and the volume rechecked and adjusted to 160 ml if necessary before conducting the test. The BFE test consists of a standard conditioning cycle with blade tip speed of 100 mm/s in a 5° C. negative upward helical path followed by a test cycle with a downward transverse tip speed set at 100 mm/s and a 10° negative helix.

The specific pore volume of a packed body of the granular material may, for example, be at least about 3 cc/g, or at least about 4 cc/g or at least about 5 cc/g. Typically, products of the invention have little, if any, pore volume in pores smaller than 0.1 μm or larger than 100 μm. The majority of the pore volume, for example at least 70% of the pore volume may be in pores larger than 1 μm and smaller than 100 μm. At least 40% of the pore volume may be in pores larger than 10 μm and smaller than 100 μm. Pore volume may be measured by mercury porosimetry using a CE Instruments Model “Pascal 240” mercury porosimeter. The method involves evacuation of the sample placed in a dilatometer, which is subsequently filled with mercury. Pressure is applied to the filled dilatometer and the mercury intrudes first into the intra-particle pores between granules and the hollow voids of the particles, and then into the pores of the granules within the sample under test. The volume of mercury intruded is determined by a precision capacitive electrode and the pore diameter calculated from the applied pressure according to the Washburn equation. The contact angle for porosimetery was 140°, and the pressure typically 0.012 MPa to 200 MPa. The average pore diameter of a packed body of the granular material may be of the order of 5-15 μm, for example about 10 μm. Typically, the average pore diameter of the granules (excluding intra-particle pores and the hollow void formed with the granules) is of the order of 1-3 μm, for example about 2 μm. The mineral may, for example, particularly have a pore volume as described where the mineral is diatomaceous earth.

In certain embodiments, the mineral has a surface area equal to or greater than about 1 m²/g. For example, the mineral may have a surface area equal to or greater than about 2 m²/g or equal to or greater than about 5 m²/g or equal to or greater than about 10 m²/g or equal to or greater than about 20 m²/g or equal to or greater than about 30 m²/g or equal to or greater than about 40 m²/g or equal to or greater than about 50 m²/g or equal to or greater than about 60 m²/g or equal to or greater than about 70 m²/g. For example, the mineral may have a surface area ranging from about 1 m²/g to about 100 m²/g or from about 2 m²/g to about 90 m²/g or from about 5 m²/g to about 80 m²/g.

The surface area of the samples may be determined according to the BET method by the quantity of nitrogen adsorbed on the surface of said particles so to as to form a monomolecular layer completely covering said surface (measurement according to the BET method, AFNOR standard X11-621 and 622 or ISO 9277). In certain embodiments, specific surface is determined in accordance with ISO 9277, or any method equivalent thereto.

In certain embodiments, the mineral has a L whiteness value equal to or greater than about 80. For example, the mineral may have a L whiteness value equal to or greater than about 82 or equal to or greater than about 84 or equal to or greater than about 85 or equal to or greater than about 86 or equal to or greater than about 88 or equal to or greater than about 90 or equal to or greater than about 92 or equal to or greater than about 94. For example, the mineral may have a L whiteness value ranging from about 80 to about 100 or from about 82 to about 98 or from about 84 to about 96 or from about 85 to about 95.

L, a and b may be determined using the Hunter scale collected on a Spectro/plus Spectrophotometer (Colour and Appearance Technology, Inc., Princeton, N.J.) as described in the Examples below.

In certain embodiments, the mineral may be spray-dried (i.e. the product of a spray-drying process). The spray-dried mineral product may, for example, be treated by one or more physical or chemical modification processes, such as milling, drying, air classifying, silanization and calcinations.

The spray-dried mineral may, for example, comprise substantially spherical granules. For example, greater than about 50 wt % of the spray-dried mineral, for example greater than about 60 wt %, for example greater than about 70 wt %, for example greater than about 80 wt %, for example greater than about 90 wt % of the spray-dried mineral may comprise substantially spherical granules. For example, each substantially spherical granule may have a mineral shell surrounded by a hollow core. The product may, for example, have substantially the same form after any of the physical or chemical modification processes described above, for example after calcinations.

The spray-dried mineral may, for example, further comprise a binder, which may, for example, have been included in the suspension that was spray-dried to facilitate the formation of spray-dried granules. A binder that remains associated with or in the product may be referred to as a “permanent hinder”. Examples of permanent binders are cross-linked alginates, thermosetting resins, thermoplastic resins and styrene-butadiene polymers. The binder may, for example; be present in the spray-dried mineral in an amount equal to or less than about wt %, for example equal to or less than about 8 wt %, for example equal to or less than about 6 wt %, for example equal to or less than about 5 wt %, for example equal to or less than about 4 wt %, for example equal to or less than about 3 wt %, for example equal to or less than about 2 wt %, for example equal to or less than about 1 wt %.

The spray-drying process may yield uniform, or substantially uniform, spray-dried granules, in which case the diameter of the granules will lie in the aforesaid range. The steepness of the particle size distribution curve, as characterized by the d₉₀/d₁₀ ratio, is typically at least 5, preferably at least 8. In some embodiments, the spray-dried granulate may be essentially mono-disperse.

In certain embodiments, the spray-dried mineral may have a d₁₀ ranging from about 1 to about 150 μm, for example from about 2 to about 140 μm, for example from about 2 to about 130 μm, for example from about 2 to about 120 μm. For example; the spray-dried mineral may have a d₁₀ ranging from about 1 to about 30 μm, for example from about 1 to about 25 μm, for example from about 1 to about 20 μm. For example, the spray-dried mineral may have a d₁₀ ranging from about 80 to about 150 μm, for example from about 80 to about 140 μm, for example from about 80 to about 130 μm, for example from about 80 to about 120 μm, for example from about 90 to about 110 μm.

In certain embodiments, the spray-dried mineral may have a d₅₀ ranging from about 10 to about 200 μm, for example from about 15 to about 180 μm, for example from about 20 to about 160 μm. For example, the spray-dried mineral may have a d₅₀ ranging from about 10 to about 60 μm, for example from about 15 to about 55 μm, for example from about 15 to about 50 μm, for example from about 20 to about 40 μm, for example from about 20 to about 30 μm. For example, the spray-dried mineral may have a d₅₀ ranging from about 100 to about 200 μm, for example from about 120 to about 180 μm, for example from about 130 to about 180 μm, for example from about 140 to about 170 μm, for example from about 140 to about 160 μm, for example from about 150 to about 160 μm.

In certain embodiments, the spray-dried mineral may have a d₉₀ ranging from about 20 to about 300 μm, for example from about 30 to about 290 μm, for example from about 40 to about 280 μm, for example from about 50 to about 270 μm, for example from about 50 to about 260 μm. For example, the spray-dried mineral may have a d₉₀ ranging from about 20 to about 100 μm, for example from about 30 to about 90 μm, for example from about 40 to about 80 μm, for example from about 50 to about 80 μm. For example, the spray-dried mineral may have a d₉₀ ranging from about 200 to about 300 μm, for example from about 210 to about 290 μm, for example from about 220 to about 280 μm, for example from about 230 to about 270 μm, for example from about 240 to about 260 μm.

In certain embodiments, the spray-dried mineral may have an organic acid-absorption capacity ranging from about 300 to about 400 g/100 g of the mineral, for example from about 320 to about 380 g/100 g of the mineral, for example from about 340 to about 380 g/100 g of the mineral. In certain embodiments, the spray-dried mineral may have a water-absorption capacity ranging from about 250 to about 400 g/100 g of the mineral, for example from about 250 to about 380 g/100 g of the mineral, for example from about 250 to about 360 g/100 g of the mineral.

In certain embodiments, the mineral is agglomerated (granulated) with a binder, wherein one or more smaller particles are attached to form a larger particle. The binder may, for example, be any of the binders described herein, which may also be used for spray drying. For example, the binder may be polyvinyl alcohol. For example, the binder may be partly saponified. For example, the binder may have a viscosity of about 5 cps. For example, the binder may have a % hydrolysation equal to or greater than about 80%, for example equal to or greater than about 85%, for example about 88%. For example, the binder may be Celvol 205E®, available from Sekisui. For example, the binder may be used in an amount equal to or less than about 10% based on the total weight of the mineral. For example, the binder may be used in an amount ranging from about 0.5 to about 8%, for example from about 1 to about 6%, for example from about 1 to about 5%, for example from about 1 to about 3%, based on the total weight of the mineral. For example, the binder may be used in an amount equal to or less than about 5 wt %, for example equal to or less than about 4 wt %, for example equal to or less than about 3 wt %.

The mineral may, for example, be agglomerated by any one or more of the methods described below in relation to making the composites comprising first and second silica- or silicate-based minerals. The mineral may, for example, be agglomerated by mixing the mineral with a binder, for example using an Eirich mixer or a food mixer (e.g. Hobart food mixer) or using a pelietizer such as a pan pelietizer or drum pelietizer etc. The mineral may, for example, be agglomerated by spray-drying. The mineral may, for example, be agglomerated by spray-drying the binder onto the mineral. For example, a 100 g solution of 3 wt % polyvinyl alcohol may be sprayed onto 100 g of the mineral (e.g. diatomaceous earth). The mineral may, for example, be agglomerated by precipitating the binder onto the mineral in situ.

### Methods of Spray-Drying

There is also provided a method of making any of the minerals described herein. For example, the method may involve spray-drying any of the minerals described herein. In certain embodiments, spray-drying a mineral may improve the oil and/or water-absorption properties of the mineral. Thus, there is also provided herein a method of improving the oil- and/or water-absorption properties of the mineral (e.g. a method of increasing the oil- and/or water-absorption properties (e.g. the oil- and/or water-absorption capacities) of the mineral).

Spray-drying a mineral may independently increase each of the organic acid- and/or oil- and/or water-absorption capacity of the mineral by at least about 10 g/100 g of the mineral. For example, spray-drying may independently increase each of the organic acid- and/or oil- and/or water-absorption capacity of the mineral by at least about 20 g/100 g of the mineral, for example by at least about 25 g/100 g of the mineral, for example at least about 30 g/100 g of the mineral, for example at least about 35 g/100 g of the mineral, for example at least about 40 g/100 g of the mineral, for example at least about 45 g/100 g of the mineral, for example at least about 50 g/100 g of the mineral, for example at least about 55 g/100 g of the mineral, for example at least about 60 g/100 g of the mineral. For example, spray-drying may independently increase each of the organic acid- and/or oil- and/or water-absorption capacity from about 10 to about 150 g/100 g of the mineral, for example from about 15 to about 150 g/100 g of the mineral, for example from about 20 to about 150 g/100 g of the mineral, for example from about 20 to about 120 g/100 g of the mineral, for example from about 20 to about 100 g/100 g of the mineral, for example from about 25 to about 90 g/100 g of the mineral.

Spray-drying is a method of producing a dry powder from a liquid or slurry by rapidly drying using a hot gas. The methods described herein comprise a step in which a suspension comprising particles of mineral is spray-dried. A mineral granulate is recovered. The recovered granulate may be heat treated (also referred to herein as “calcined”).

For example, the methods described herein may comprising spray-drying a suspension comprising particles of a mineral (e.g. a mineral as described herein), a liquid medium and a binder and recovering a spray-dried mineral granulate. The mineral starting material and/or the spray-dried mineral granulate may have any one or more of the characteristics described herein. For example, the spray-dried mineral granulate may have an increased organic acid- and/or oil- and/or water-absorption capacity in comparison to the organic acid- and/or oil- and/or water-absorption capacity of the mineral prior to the spray-drying step.

The suspension which is to be spray-dried is typically an aqueous suspension comprising a liquid medium and a solids portion. The liquid medium is typically water.

The suspension may further include a binder. The binder may be inorganic or organic and may comprise a solid component, as for example a latex type binder. The binder may be included in the suspension to facilitate the formation of spray-dried granules.

In an embodiment, the binder may be a temporary binder. By “temporary binder” is meant a binder which is not intended to remain in the product but acts to bind particles of the mineral together and support the spray-dried body after initial formation, which can then be subjected to one or more further treatment steps, including steps intended to impart structural rigidity to the spray-dried bodies, such as a heat treatment. Such temporary binders may thus be thermally fugitive, that is to say are removed from the spray-dried bodies on the application of sufficient heat which may vaporize or burn the binder material. Examples of suitable temporary binders are starches, carbohydrates, sugars, poly-vinyl acetates, poly-vinyl alcohols, latex, gelatines, waxes, celluloses, dextrines, thermo-plastic resins, thermo-setting resins, chlorinated hydrocarbons, gums, flours, caseins, alginates, proteins, bitumens, acrylics, epoxy resins, and urea. In embodiments of the invention, the temporary binder may be a poly vinyl alcohol binder or a latex binder. The amount of temporary binder in the suspension may be in the range of up to 10 wt % on a solids basis, for example 2-10 wt %. Where the binder is a temporary binder, the spray-dried granulate may be subjected to a heat treatment, or calcination, step in order to impart structural rigidity to the spray-dried bodies. In the heat treatment step, the temporary binder is removed, or substantially removed, from the spray-dried bodies.

In another embodiment, the binder may be a permanent binder. By “permanent binder” is meant a binder which is intended to remain in the product and provide structural strength to the spray-dried bodies without the need for a high temperature calcination step. Examples of permanent binders are cross-linked alginates, thermosetting resins, thermoplastic resins and styrene-butadiene polymers. The specific permanent binder to be used may be selected to ensure that the binder provides structural support to the aggregate without being significantly soluble in the liquid to be filtered. For example a binder which is insoluble in water would be suitable for use in a filter medium which is to be used in beer filtration.

The permanent binder may also, for example, be cross-linkable. In case such cross-linkable binders are used, a further chemical or low temperature heat treatment (for example less than 200° C.) may be required after the spray-dried bodies are formed in order to effect cross-linking. An example of a suitable cross-linkable binder is a copolymer of a vinyl acetate and an acrylic ester, such as Vinnapas AN214 from Wacker Chemie. It is to be appreciated that permanent binders used in the present invention may be thermally fugitive, if organic in nature. However, a distinction between a temporary binder and a permanent binder which is thermally fugitive is that a permanent binder is capable of fixing the aggregated structure produced during the spray-drying step, without the need for a calcination treatment.

Other permanent binders which are not thermally fugitive may be used. Such binders are inorganic-based. Examples include cements, pozzolanic materials, silcates, waterglass, gypsums, bentonites, and borates. Also included are aluminate binders, including alkali metal aluminate binders such as sodium aluminate, potassium aluminate or lithium aluminate, and alkaline earth metal aluminate binders, such as calcium aluminate and magnesium aluminate. An advantage of using a permanent binder is that a calcination step can be avoided.

The solids portion of the suspension comprises the particulate mineral component together with one or more optional additional inorganic components and one or more optional organic solid components.

The inorganic solids content of the suspension is dependent on the spray-drying method to be used, which is discussed in more detail below, and the size of spray-dried granules desired. Typically, however, in order to have a viscosity suitable for spray-drying, the suspension should have an inorganic solids content of at least 5%, for example at least 10%, for example at least 15% by weight, based on the weight of the suspension, and may have an inorganic solids content of up to 30%, or 25% or 20%, based on the weight of the suspension. Typically, the solids content will be in the range of 15-25% by weight, based on the weight of the suspension.

The optional inorganic component may comprise one or more particulate inorganic mineral in addition to the mineral as described herein; and/or one or more suitable fluxing agent. The optional organic solids component may be the solids component of an organic binder.

A small amount of an additional inorganic mineral component, for example 20% or less, based on the total weight of the inorganic solids in the suspension, for example 10% or less or 5% or less, based on the total weight of the inorganic solids present in the suspension may be included in the suspension that is spray-dried. This will result in spray-dried granules including the additional inorganic mineral component, for example in the outer wall thereof. This may be used to adjust the properties of the spray-dried granules, for example strength and/or permeability. Examples of additional inorganic mineral components are natural or synthetic silicate or aluminosilicate materials, unimproved diatomaceous earth, saltwater diatomaceous earth, expanded perlite, pumicite, natural glass, cellulose, activated charcoal, feldspars, nepheline syenite, sepiolite, zeolite, and clay. Examples of clay minerals are halloysite, kaolinite and bentonite.

A fluxing agent is an optional additional component of the suspension that is spray-dried. A fluxing agent may be necessary where the spray-dried granules are to be calcined (so-called “flux-calcining”). The presence of at least one fluxing agent during calcination may reduce the temperature at which mineral particles in the wall of the spray-dried bodies are caused to be sintered together.

Suitable agents as the fluxing agent are any now known to those skilled in the art or which may hereafter be discovered. In one embodiment, the fluxing agent is sodium carbonate (soda ash, Na₂CO₃). In another embodiment, the fluxing agent is sodium hydroxide (NaOH). In a further embodiment, the at least one fluxing agent is sodium chloride (NaCl). In yet another embodiment, the at least one fluxing agent is potassium carbonate (K₂CO₃). In yet a further embodiment, the at least one fluxing agent is sodium borate (Na₂B₄O₇).

In one embodiment, the fluxing agent is at least one salt of at least one alkali metal in Group IA. In another embodiment, the fluxing agent is at least one salt of at least one alkali metal. In a further embodiment, the at least one alkali metal is sodium. In yet another embodiment, the at least one alkali metal is chosen from alkali metals having a larger atomic radius than that of sodium. In yet a further embodiment, the at least one alkali metal is potassium. In still another embodiment, the at one alkali metal is rubidium.

The at least one fluxing agent is added to the suspension before spray-drying; as a result, the fluxing agent is located within the wall of the spray-dried granules at locations where it is readily able to provide its fluxing function.

The fluxing agent may be present in the suspension in an amount of less than about 8% based on the total weight of inorganic solids in the suspension, or in an amount of less than about 7%, or an amount of less than about 6%, or in amount of less than about 5%, or in amount of less than about 4%, or in amount of less than about 3%, or in amount less than about 2%. In another embodiment, the suspension contains from about 0.5% to about 10% fluxing agent, based on the total weight of inorganic solids in the suspension.

In some embodiments where the spray-dried granules are flux-calcined, the at least one fluxing agent may undergo a chemical decomposition reaction. In one embodiment of such a chemical decomposition, at least one fluxing agent containing sodium bonds with diatom silica present in the at least one feed material to form sodium silicate, expelling carbon dioxide gas in the process. In another embodiment, at least one fluxing agent containing at least one alkaline metal bonds with diatom silica present in the at least one feed material to form at least one alkaline metal silicate.

The suspension may be spray-dried in a manner which is known per se. The suspension may be fed to the inlet of a spray-dryer and spray-dried material is discharged from the atomiser.

Spray-drying may also be carried out using a nozzle atomiser or fountain spray-drying technique, in which the slurry is sprayed upwards from the cone of the drying chamber. This allows drying to take place during the complete flight-arc of the droplets before they return to the bottom of the dryer, providing a coarser, more free-flowing powder.

Another type of spray-dryer which may be used in the invention is one which employs a “rotating wheel” or “spinning disc” atomiser.

One example of a suitable spray-drying apparatus is a Niro Minor spray dryer unit. This machine has a drying chamber 800 mm in diameter, 600 mm cylindrical height being conical based and is fitted with an air driven disc type atomiser. The atomiser may be run at a speed of 30,000 rpm. Drying may be carried out using an inlet-air temperature of 300° C. Slurry is fed via a peristaltic pump to the atomiser at a rate selected to maintain the required outlet temperature (typically 110 to 120° C.).

In one example of a method of spray-drying, an inlet temperature between 350 and 400° C. and an outlet temperature between 110 and 120° C. was used.

The inorganic solids content of the suspension is dependent on the spray-drying method to be used, which is discussed in more detail below, and the size of spray-dried granules desired, Typically, however, the suspension will have an inorganic solids content of the order of 5 to 30 wt %, for example 15 to 25 wt %.

The heat treatment, also referred to herein as a calcination treatment may be carried out at a suitable temperature to cause mineral particles in the wall of the spray-dried bodies to be sintered together and thus result in a body which is resistant to crushing. The maximum calcination temperature may be for example at least 500° C., or at least 600° C., or at least 700° C., or at least 800° C., or at least 900° C., In order to avoid destroying the fine structure of the spray-dried bodies and incurring additional cost, the maximum calcination temperature is typically less than 1200° C., for example less than 1100° C. or less than 1000° C.

The duration of calcination can be determined empirically depending on the desired outcome. However, typically calcination may be carried out such that the duration at peak temperature is less than four hours, or less than three hours, or less than two hours, or less than one hour. In an embodiment, calcination may be carried out by “flash” calcination, in which the calcination is conducted very rapidly. Calcination may be carried out in a batch process, or in a continuous process. A suitable continuous process may use a rotary tube furnace, in which the uncalcined feed material is continuously passed through a heated zone maintained at the appropriate temperature. In embodiments, the calcination may be carried out by increasing the calcination temperature at a rate of, for example, between 1 and 50° C. per minute, for example 1 to 10° C. per minute, up to the final, maximum temperature and then cooled at a rate of, for example, 1 to 50° C. per minute, for example 5 to 20° C. per minute, to room temperature.

The calcined, spray-dried granulate has substantially the same particle size distribution as the uncalcined starting material.

### Mineral Composites

There is provided herein mineral composites comprising a first silica- or silicate-based mineral and a second, different, silica- or silicate-based mineral.

Each of the first and second silica- or silicate-based mineral components may be as described above, including any embodiment or combination of embodiments disclosed herein.

For example, one or both of the silica- or silicate-based mineral components may be derived from a natural mineral or may be synthetic. The invention may tend to be described in terms of two silica- or silicate-based mineral components that are naturally-derived. However, the present invention should not be construed as being limited as such.

For example, one or both of the silica- or silicate-based mineral components may be silica-based minerals. The invention may tend to be described in terms of one silica-based mineral component and one silicate-based mineral component. However, the present invention should not be construed as being limited as such.

For example, in certain embodiments one of the mineral components is perlite (e.g. expanded perlite) or diatomaceous earth. For example, in certain embodiments, one of (e.g. the other of) the mineral components is calcium silicate (e.g. calcium silicate derived from a natural mineral), For example, in certain embodiments, the first silica- or silicate-based mineral is perlite and the second silica- or silicate-based mineral is calcium silicate.

For example, one or more of the mineral components may be spray-dried.

In certain embodiments, the first and second silica- or silicate-based minerals are co-agglomerated. For example, the first and second silica- or silicate-based mineral may be co-agglomerated with at least one binder. The binder may, for example, be a precipitated binder. The binder may, for example, be a silica binder. For example, the binder may be an alkali silica binder. For example, the binder may be sodium silicate and/or potassium silicate. Without wishing to be bound by theory, co-agglomeration of the first and second silica- or silicate-based minerals may result in the first and second mineral components attaching to one another to form larger particles relative to a blend of the first and second mineral components.

The first and second silica- or silicate-based minerals may be present in the composition in a ratio ranging from 1:99 to 99:1. For example, the first and second silica- or silicate-based minerals may be present in the composition in a ratio ranging from 1:50 to 50:1, for example from 1:20 to 20:1, for example from 1:10 to 10:1, for example from 1:5 to 5:1, for example from 1:3 to 3:1, For example, the first and second silica- or silicate-based minerals may be present in the composition in a ratio of about 1:1.

In certain embodiments, the mineral composite further comprises a third silica- or silicate-based mineral. In certain embodiments, the mineral composite further comprises a fourth silica- or silicate-based mineral. In certain embodiments, the mineral composite further comprises a fifth silica- or silicate-based mineral. The optional third, fourth and fifth silica- or silicate-based minerals may be according to any aspect or embodiments described above, including all combinations thereof.

The mineral composite may also comprise one or more further minerals that are not silica- or silicate-based minerals (e.g. calcium carbonate). Where a further non-silica or silicate-based mineral is present, it is present in an amount ranging from about 1% to about 20% by weight of the total mineral in the composite, for example from about 1% to about 15% or from about 1% to about 10% or from about 1% to about 5% by weight of the total mineral in the composite.

The mineral composite may have a d₁₀ ranging from about 0.1 to about 30 μm. For example, the mineral composite may have a d₁₀ ranging from about 0.1 to about 20 μm, for example from about 0.1 to about 10 μm, for example from about 1 to about 10 μm, for example from about 1 to about 8 μm, for example from about 1 to about 6 μm, for example from about 2 to about 8 μm, for example from about 2 to about 6 μm, for example from about 4 to about 8 μm, for example from about 4 to about 6 μm.

The mineral composite may, for example, have a d₅₀ ranging from about 5 to about 50 μm. For example, the mineral composite may have a d₅₀ ranging from about 5 to about 40 μm, for example from about 5 to about 30, for example from about 10 to about 30 μm, for example from about 15 to about 25 μm.

The mineral composite may, for example, have a d₉₀ ranging from about 20 to about 80 μm. For example, the mineral composite may have a d₉₀ ranging from about 20 to about 70 μm, for example from about 20 to about 60 μm, for example from about 30 to about 60 μm, for example from about 30 to about 50 μm, for example from about 30 to about 40 μm.

In certain embodiments, the mineral composite (i.e. the composition comprising a first and second silica- or silicate-based mineral) has an organic acid-absorption capacity equal to or greater than about 50 g/100 g of total mineral in the composite. For example, the mineral composite may have an organic acid-absorption capacity equal to or greater than about 100 g/100 g of total mineral in the composite, for example equal to or greater than about 120 g/100 g of total mineral in the composite, for example equal to or greater than about 140 g/100 g of total mineral in the composite, for example equal to or greater than about 150 g/100 g of total mineral in the composite, for example equal to or greater than about 160 g/100 g of total mineral in the composite, for example equal to or greater than about 170 g/100 g of total mineral in the composite, for example equal to or greater than about 180 g/100 g of total mineral in the composite, for example equal to or greater than about 190 g/100 g of total mineral in the composite, for example equal to or greater than about 200 g/100 g of total mineral in the composite, for example equal to or greater than about 210 g/100 g of total mineral in the composite, for example equal to or greater than about 220 g/100 g of total mineral in the composite, for example equal to or greater than about 230 g/100 g of total mineral in the composite, for example equal to or greater than about 240 g/100 g of total mineral in the composite, for example equal to or greater than about 250 g/100 g of total mineral in the composite, for example equal to or greater than about 260 g/100 g of total mineral in the composite, for example equal to or greater than about 270 g/100 g of total mineral in the composite, for example equal to or greater than about 280 g/100 g of total mineral in the composite, for example equal to or greater than about 290 g/100 g of total mineral in the composite, for example equal to or greater than about 300 g/100 g of total mineral in the composite. For example, the composite may have an organic acid-absorption capacity equal to or greater than about 310 g/100 g of total mineral in the composite, for example equal to or greater than about 320 g/100 g of total mineral in the composite, for example equal to or greater than about 330 g/100 g of total mineral in the composite, for example equal to or greater than about 340 g/100 g of total mineral in the composite, for example equal to or greater than about 350 g/100 g of total mineral in the composite. For example, the composite may have an organic acid-absorption capacity equal to or greater than about 400 g/100 g of total mineral in the composite, for example equal to or greater than about 450 g/100 g of total mineral in the composite, for example equal to or greater than about 500 g/100 g of total mineral in the composite.

In certain embodiments, the composite may have an organic acid-absorption capacity ranging from about 50 to about 800 g/100 g of total mineral in the composite, for example from about 100 to about 800 g/100 g of total mineral in the composite, for example from about 200 to about 800 g/100 g of total mineral in the composite. For example, the composite may have an organic acid-absorption capacity ranging from about 220 to about 800 g/100 g of total mineral in the composite, for example from about 220 to about 600 g/100 g of total mineral in the composite, for example from about 270 to about 800 g/100 g of total mineral in the composite, for example from about 270 to about 600 g/100 g of total mineral in the composite, for example from about 300 to about 800 g/100 g of total mineral in the composite, for example from about 300 to about 600 g/100 g of total mineral in the composite, for example from about 300 to about 500 g/100 g of total mineral in the composite. The present invention may tend to be discussed in terms of composites having an organic acid-absorption capacity equal to or greater than about 200 g/100 g of total mineral in the composite (e.g. from about 200 to about 400 g/100 g of total mineral in the composite). However, the invention should not be construed as being limited to such embodiments.

In certain embodiments, the mineral composite (i.e. the composition comprising a first and second silica- or silicate-based mineral) has an oil-absorption capacity equal to or greater than about 50 g/100 g of total mineral in the composite. For example, the mineral composition may have an oil-absorption capacity equal to or greater than about 100 g/100 g of total mineral in the composite, for example equal to or greater than about 120 g/100 g of total mineral in the composite, for example equal to or greater than about 140 g/100 g of total mineral in the composite, for example equal to or greater than about 150 g/100 g of total mineral in the composite, for example equal to or greater than about 160 g/100 g of total mineral in the composite, for example equal to or greater than about 170 g/100 g of total mineral in the composite, for example equal to or greater than about 180 g/100 g of total mineral in the composite, for example equal to or greater than about 190 g/100 g of total mineral in the composite, for example equal to or greater than about 200 g/100 g of total mineral in the composite, for example equal to or greater than about 210 g/100 g of total mineral in the composite, for example equal to or greater than about 220 g/100 g of total mineral in the composite, for example equal to or greater than about 230 g/100 g of total mineral in the composite, for example equal to or greater than about 240 g/100 g of total mineral in the composite, for example equal to or greater than about 250 g/100 g of total mineral in the composite, for example equal to or greater than about 260 g/100 g of total mineral in the composite, for example equal to or greater than about 270 g/100 g of total mineral in the composite, for example equal to or greater than about 280 g/100 g of total mineral in the composite, for example equal to or greater than about 290 g/100 g of total mineral in the composite, for example equal to or greater than about 300 g/100 g of total mineral in the composite. For example, the composite may have an oil-absorption capacity equal to or greater than about 310 g/100 g of total mineral in the composite, for example equal to or greater than about 320 g/100 g of total mineral in the composite, for example equal to or greater than about 330 g/100 g of total mineral in the composite, for example equal to or greater than about 340 g/100 g of total mineral in the composite, for example equal to or greater than about 350 g/100 g of total mineral in the composite. For example, the composite may have an oil-absorption capacity equal to or greater than about 400 g/100 g of total mineral in the composite, for example equal to or greater than about 450 g/100 g of total mineral in the composite, for example equal to or greater than about 500 g/100 g of total mineral in the composite.

In certain embodiments, the composite may have an oil-absorption capacity ranging from about 50 to about 800 g/100 g of total mineral in the composite, for example from about 100 to about 800 g/100 g of total mineral in the composite, for example from about 200 to about 800 g/100 g of total mineral in the composite. For example, the composition may have an oil-absorption capacity ranging from about 220 to about 800 g/100 g of total mineral in the composite, for example from about 220 to about 600 g/100 g of total mineral in the composite, for example from about 270 to about 800 g/100 g of total mineral in the composite, for example from about 270 to about 600 g/100 g of total mineral in the composite, for example from about 300 to about 800 g/100 g of total mineral in the composite, for example from about 300 to about 600 g/100 g of total mineral in the composite, for example from about 300 to about 500 g/100 g of total mineral in the composite. The present invention may tend to be discussed in terms of composites having an oil-absorption capacity equal to or greater than about 200 g/100 g of total mineral in the composite (e.g. from about 200 to about 400 g/100 g of total mineral in the composite), However, the invention should not be construed as being limited to such embodiments.

In certain embodiments, the composite has a water-absorption capacity equal to or greater than about 50 g/100 g of the total mineral in the composite. For example, the composite may have a water-absorption capacity equal to or greater than about 100 g/100 g of the total mineral in the composite, for example equal to or greater than about 120 g/100 g of the total mineral in the composite, for example equal to or greater than about 140 g/100 g of the total mineral in the composite, for example equal to or greater than about 150 g/100 g of the total mineral in the composite, for example equal to or greater than about 160 g/100 g of the total mineral in the composite, for example equal to or greater than about 170 g/100 g of the total mineral in the composite, for example equal to or greater than about 180 g/100 g of the total mineral in the composite, for example equal to or greater than about 190 g/100 g of the total mineral in the composite, for example equal to or greater than about 200 g/100 g of the total mineral in the composite, for example equal to or greater than about 210 g/100 g of the total mineral in the composite, for example equal to or greater than about 220 g/l 00 g of the total mineral in the composite, for example equal to or greater than about 230 g/100 g of the total mineral in the composite, for example equal to or greater than about 240 g/100 g of the total mineral in the composite, for example equal to or greater than about 250 g/100 g of the total mineral in the composition, for example equal to or greater than about 260 g/100 g of the total mineral in the composite, for example equal to or greater than about 270 g/100 g of the total mineral in the composite, for example equal to or greater than about 280 g/100 g of the total mineral in the composite, for example equal to or greater than about 290 g/100 g of the total mineral in the composite, for example equal to or greater than about 300 g/100 g of the total mineral in the composite. For example, the composite may have a water-absorption capacity equal to or greater than about 310 g/100 g of the total mineral in the composite, for example equal to or greater than about 320 g/100 g of the total mineral in the composite, for example equal to or greater than about 330 g/100 g of the total mineral in the composite, for example equal to or greater than about 340 g/100 g of the total mineral in the composite, for example equal to or greater than about 350 g/100 g of the total mineral in the composite. For example, the composite may have a water-absorption capacity equal to or greater than about 400 g/100 g of the total mineral in the composite, for example equal to or greater than about 450 g/100 g of the total mineral in the composite, for example equal to or greater than about 500 g/100 g of the total mineral in the composite.

In certain embodiments, the composite may have a water-absorption capacity ranging from about 50 to about 800 g/100 g of the total mineral in the composite, for example from about 100 to about 800 g/100 g of the total mineral in the composite, for example from about 200 to about 800 g/100 g of the total mineral in the composite. For example, the composite may have a water-absorption capacity ranging from about 220 to about 800 g/100 g of the total mineral in the composite, for example from about 220 to about 600 g/100 g of the total mineral in the composite, for example from about 270 to about 800 g/100 g of the total mineral in the composite, for example from about 270 to about 600 g/100 g of the total mineral in the composite, for example from about 300 to about 800 g/100 g of the total mineral in the composite, for example from about 300 to about 600 g/100 g of the total mineral in the composite, for example from about 300 to about 500 g/100 g of the total mineral in the composite. The present invention may tend to be discussed in terms of composite having a water-absorption capacity equal to or greater than about 200 g/100 g of the total mineral in the composite (e.g. from about 200 to about 500 g/100 g of the total mineral in the composite). However, the invention should not be construed as being limited to such embodiments.

In certain embodiments, the composite independently has an organic acid- and/or oil- and/or water-absorption capacity that is each greater than the respective mean organic acid- and/or oil- and/or water-absorption capacity of the silica- or silicate-based mineral components (e.g. the first and second silica- or silicate-based mineral components). For example, the composite may independently have an organic acid- and/or oil- and/or water-absorption capacity that is at least about 20 g/100 g of total mineral in the composite greater than the respective mean organic acid- and/or oil- and/or water-absorption capacity of the silica- or silicate-based mineral compositions. For example, the composite may independently have an organic acid- and/or oil- and/or water-absorption capacity that is at least about 25 g/100 g of total mineral in the composite, for example at least about 30 g/10 g of total mineral in the composite, for example at least about 35 g/100 g of total mineral in the composite, for example at least about 40 g/100 g of total mineral in the composite, for example at least about 45 g/100 g of total mineral in the composite, for example at least about 50 g/100 g of total mineral in the composite, greater than the respective mean organic acid- and/or oil- and/or water-absorption capacity of the silica- or silicate-based mineral components.

In certain embodiments, the composite (i.e. comprising first and second silica- or silicate-based minerals) is the form of a free-flowing granulate. For example, the composite may have a BFE equal to or less than about 1200 mJ. For example, the composite may have a BFE equal to or less than about 1100 mJ, for example equal to or less than about 1000 mJ, for example equal to or less than about 800 mJ, for example equal to or less than about 700 mJ, for example equal to or less than about 600 mJ.

In certain embodiments, the composite is in the form of a free-flowing granulate even at relatively high oil and/or water contents. For example, the composite may be in the form of a free-flowing granulate at a liquid (e.g. organic acid and/or oil and/or water) content of at least about 140 g/100 g of the composite. For example, the composite may be in the form of a free-flowing granulate at a liquid content of at least about 150 g/100 g of the composite, for example at least about 160 g/100 g of the composite, for example at least about 170 g/100 g of the composite, for example at least about 180 g/100 g of the composite, for example at least about 190 g/100 g of the composite, for example at least about 200 g/100 g of the composite, for example at least about 210 g/100 g of the composite, for example at least about 220 g/100 g of the composite, for example at least about 230 g/100 g of the composite, for example at least about 240 g/100 g of the composite, for example at least about 250 g/100 g of the composite, for example at least about 260 g/100 g of the composite, for example at least about 270 g/100 g of the composite, for example at least about 280 g/100 g of the composite, for example at least about 290 g/100 g of the composite, for example at least about 300 g/100 g of the composite. For example, the composite may be in the form of a free-flowing granulate at a liquid content ranging from about 140 to about 600 g/100 g of the composite, for example from about 150 to about 550 g/100 g of the composite, for example from about 160 to about 500 g/100 g of the composite.

In certain embodiments, the composite has a base flow energy (BFE) equal to or less that is equal to or less than about 1200 mJ when the composite has a liquid content of 200 g/100 g of the composite. For example, the composite may have a BFE equal to or less than about 1100 mJ when the composite has a liquid content of 200 g/100 g of the composite, for example equal to or less than about 1000 mJ when the composite has a liquid content of 200 g/100 g of the composite, for example equal to or less than about 900 mJ when the composite has a liquid content of 200 g/100 g of the composite, for example equal to or less than about 800 mJ when the composite has a liquid content of 200 g/100 g of the composite. For example, the composite may have a BFE ranging from about 200 to about 1200 mJ when the composite has a liquid content of 200 g/100 g of the composite, for example from about 300 to about 1100 mJ when the composite has a liquid content of 200 g/100 g of the composite, for example from about 400 to about 1000 mJ when the composite has a liquid content of 200 g/100 g of the composite, for example from about 400 to about 800 mJ when the composite has a liquid content of 200 g/100 g of the composite.

In certain embodiments, the composite has a base flow energy (BFE) equal to or less that is equal to or less than about 1200 mJ when the composite has an organic acid content of 185 g/100 g of the composite. For example, the composite may have a BFE equal to or less than about 1100 mJ when the composite has an organic acid content of 185 g/100 g of the composite, for example equal to or less than about 1000 mJ when the composite has an organic acid content of 185 g/100 g of the composite, for example equal to or less than about 900 mJ when the composite has an organic acid content of 185 g/100 g of the composite, for example equal to or less than about 800 mJ when the composite has an organic acid content of 185 g/100 g of the composite. For example, the composite may have a BFE ranging from about 200 to about 1200 mJ when the composite has an organic acid content of 185 g/100 g of the composite, for example from about 300 to about 1100 mJ when the composite has an organic acid content of 185 g/100 g of the composite, for example from about 400 to about 1000 mJ when the composite has an organic acid content of 185 g/l 00 g of the composite, for example from about 400 to about 800 mJ when the composite has an organic acid content of 185 g/100 g of the composite.

In certain embodiments, the composite has a base flow energy (BFE) that is equal to or less than about 1200 mJ when the composite has a water content of 150 g/100 g of the composite. For example, the composite may have a BFE equal to or less than about 1100 mJ when the composite has a water content of 150 g/100 g of the composite, for example equal to or less than about 1000 mJ when the composite has a water content of 150 g/100 g of the composite, for example equal to or less than about 900 mJ when the composite has a water content of 150 g/100 g of the composite, for example equal to or less than about 800 mJ when the composite has a water content of 150 g/100 g of the composite. For example, the composite may have a BFE ranging from about 200 to about 1200 mJ when the composite has a water content of 150 g/100 g of the composite, for example from about 300 to about 1100 mJ when the composite has a water content of 150 g/100 g of the composite, for example from about 400 to about 1000 mJ when the composite has a water content of 150 g/100 g of the composite, for example from about 400 to about 800 mJ when the composition has a water content of 150 g/100 g of the composite.

In certain embodiments, the mineral composite has a surface area equal to or greater than about 1 m²/g. For example, the mineral composite may have a surface area equal to or greater than about 2 m²/g or equal to or greater than about 5 m²/g or equal to or greater than about 10 m²/g or equal to or greater than about 15 m²/g or equal to or greater than about 20 m²/g or equal to or greater than about 25 m²/g or equal to or greater than about 30 m²/g or equal to or greater than about 35 m²/g or equal to or greater than about 40 m²/g or equal to or greater than about 45 m²/g or equal to or greater than about 50 m²/g. For example, the mineral composite may have a surface area ranging from about 1 m²/g to about 100 m²/g or from about 2 m²/g to about 90 m²/g or from about or from about 5 m²/g to about 50 m²/g or from about 10 m²/g to about 40 m²/g.

The surface area of the mineral composite may be determined using the method described above.

In certain embodiments, the mineral composite has a L whiteness value equal to or greater than about 80. For example, the mineral composite may have a L whiteness value equal to or greater than about 82 or equal to or greater than about 84 or equal to or greater than about 85 or equal to or greater than about 86 or equal to or greater than about 88. For example, the mineral composite may have a L whiteness value ranging from about 80 to about 100 or from about 80 to about 95 or from about 80 to about 90.

L, a and b may be determined using the method described herein.

### Methods of Making the Mineral Composites

There is provided herein a method of making the mineral composites disclosed herein. In certain embodiments, the method comprises combining a first silica- or silicate-based mineral with a second silica- or silicate-based mineral that is different from the first mineral and any other optional components of the composite.

In certain embodiments, the first and second mineral components are combined by mixing and/or blending by any technique known to those skilled in the art.

In certain embodiments, the first and second mineral components are co-agglomerated. The co-agglomeration may, for example, comprise adding a binder to the first and/or second silica- or silicate-based minerals or adding a binder to a blend of the first and second silica- or silicate-based minerals.

The binder may, for example, be any of the binders described herein, which may also be used for spray drying. For example, the binder may be polyvinyl alcohol. For example, the binder may be partly saponified. For example, the binder may have a viscosity of about 5 cps. For example, the binder may have a %© hydrolysation equal to or greater than about 80%, for example equal to or greater than about 85%, for example about 88%. For example, the binder may be Celvol 205E®, available from Sekisui. For example, the binder may be used in an amount equal to or less than about 10% based on the total weight of the mineral. For example, the binder may be used in an amount ranging from about 0.5 to about 8%, for example from about 1 to about 6%, for example from about 1 to about 5%, for example from about 1 to about 3%, based on the total weight of the mineral. For example, the binder may be used in an amount equal to or less than about 5 wt %, for example equal to or less than about 4 wt %, for example equal to or less than about 3 wt %.

In certain embodiments, the binder may, for example, be a precipitated binder. The binder may, for example, be a silica binder. For example, the binder may be an alkali silica binder. For example, the binder may be sodium silicate and/or potassium silicate. Without wishing to be bound by theory, co-agglomeration of the first and second silica- or silicate-based minerals may result in the first and second mineral components attaching to one another to form larger particles relative to a blend of the first and second mineral components.

In certain embodiments, the method comprises preparing an aqueous solution of the binder (e.g. silica binder) and contacting the aqueous solution with the first and/or second silica- or silicate-based mineral or a blend of the first and second silica- or silicate-based minerals. One or more agglomerations may be performed, for example, when multiple binders (e.g. multiple silica binders) and/or multiple minerals or mineral blends are used.

In some embodiments, contacting includes mixing the binder solution with a blend of the minerals, in some embodiments, the mixing includes agitation. In some embodiments, the mineral blend is mixed sufficiently to at least substantially uniformly distribute the binder solution among the agglomeration points of contact of the mineral components. In some embodiments, the mineral blend and the binder solution are mixed with sufficient agitation to at least substantially uniformly distribute the binder solution among the agglomeration points of contact of the blend of the mineral components without damaging the structure of the mineral components. In some embodiments, the contacting includes low-shear mixing. The mineral may, for example, be agglomerated by mixing the mineral with a binder, for example using an Eirich mixer or a food mixer (e.g. Hobart food mixer) or using a pelletizer such as a pan pelletizer or drum pelletizer etc.

In some embodiments, mixing occurs for about one hour. In other embodiments, mixing occurs for less than about one hour. In further embodiments, mixing occurs for about 30 minutes. In yet other embodiments, mixing occurs for about 20 minutes. In still further embodiments, mixing occurs for about 10 minutes.

In some embodiments, mixing occurs at about room temperature (i.e., from about 20° C. to about 23° C.). In other embodiments, mixing occurs at a temperature of from about 20° C. to about 50° C. In further embodiments, mixing occurs at a temperature of from about 30° C. to about 45° C. In still other embodiments, mixing occurs at a temperature of from about 35° C. to about 40° C.

According to some embodiments, contacting includes spraying the mineral blend of with at least one binder solution. In some embodiments, the spraying is intermittent. In other embodiments, the spraying is continuous. In further embodiments, spraying includes mixing the blend while spraying with the at least one binder solution, for example, to expose different agglomeration points of contacts to the spray. In some embodiments, such mixing is intermittent. In other embodiments, such mixing is continuous. In some embodiments, the a mixture of mineral blend and binder is spray-dried together.

In some embodiments, the at least one binder is present in the binder solution in an amount from less than about 40% by weight, relative to the weight of the at least one binder solution. In some embodiments, the at least one binder ranges from about 1% to about 10% by weight. In further embodiments, the at least one binder ranges from about 1% to about 5% by weight.

The at least one aqueous solution of the at least one binder may be prepared with water. In some embodiments, the water is deionized water. In some embodiments, the water is ultrapure water. In further embodiments, the water has been treated to remove or decrease the levels of metals, toxins, and/or other undesirable elements before it is contacted with the at least one binder.

The amount of at least one aqueous solution contacted with the mineral blend may range from about 0.25 parts to about 1.5 parts of aqueous solution to one part blend. In some embodiments, about 1 part aqueous solution is contacted with about 1 part blend.

Before and/or after the agglomeration, the mineral components may be subjected to at least one classification step. For example, before and/or after at least one heat treatment, the mineral components may, in some embodiments, be subjected to at least one classification step. In some embodiments, the particle size of the mineral components is adjusted to a suitable or desired size using any one of several techniques well known in the art. In some embodiments, the mineral components are subjected to at least one mechanical separation to adjust the powder size distribution. Appropriate mechanical separation techniques are well known to the skilled artisan and include, but are not limited to, milling, grinding, screening, extrusion, triboelectric separation, liquid classification, aging, and air classification.

The mineral components and/or co-agglomerated mineral composite may be subjected to at least one heat treatment. Appropriate heat treatment processes are well-known to the skilled artisan, and include those now known or that may hereinafter be discovered. In some embodiments, the at least one heat treatment decreases the amount of organics and/or volatiles in the heat-treated mineral composition. In some embodiments, the at least one heat treatment includes at least one calcination. In some embodiments, the at least one heat treatment includes at least one flux calcination. In some embodiments, the at least one heat treatment includes at least one roasting.

Calcination may be conducted according to any appropriate process now known to the skilled artisan or hereafter discovered. In some embodiments, calcination is conducted at temperatures below the melting point of the mineral(s). In some embodiments, calcination is conducted at a temperature ranging from about 600° C. to about 1100° C. In some embodiments, the calcination temperature ranges from about 600° C. to about 700° C. In some embodiments, the calcination temperature ranges from about 700° C. to about 800° C. In some embodiments, the calcination temperature ranges from about 800° C. to about 900° C. In some embodiments, the calcination temperature is chosen from the group consisting of about 600° C., about 700° C., about 800° C., about 900° C., about 1000° C., and about 1100° C., Heat treatment at a lower temperature may result in an energy savings over other processes for the preparation of mineral composites.

Flux calcination includes conducting at least one calcination in the presence of at least one fluxing agent. Flux calcination may be conducted according to any appropriate process now known to the skilled artisan or hereafter discovered. In some embodiments, the at least one fluxing agent is any material now known to the skilled artisan or hereafter discovered that may act as a fluxing agent. In some embodiments, the at least one fluxing agent is a salt including at least one alkali metal. In some embodiments, the at least one fluxing agent is chosen from the group consisting of carbonate, silicate, chloride, and hydroxide salts. In other embodiments, the at least one fluxing agent is chosen from the group consisting of sodium, potassium, rubidium, and cesium salts. In still further embodiments, the at least one fluxing agent is chosen from the group consisting of sodium, potassium, rubidium, and cesium carbonate salts.

Roasting may be conducted according to any appropriate process now known to the skilled artisan or hereafter discovered. In some embodiments, roasting is a calcination process conducted at a generally lower temperature that helps to avoid formation of crystalline mineral (e.g. crystalline silica) in the mineral (e.g. diatomaceous earth and/or natural glass). In some embodiments, roasting is conducted at a temperature ranging from about 450° C. to about 900° C. In some embodiments, the roasting temperature ranges from about 500° C. to about 800° C. In some embodiments, the roasting temperature ranges from about 600° C. to about 700° C. In some embodiments, the roasting temperature ranges from about 700° C. to about 900° C. In some embodiments, the roasting temperature is chosen from the group consisting of about 450° C., about 500° C., about 600° C., about 700° C., about 800° C., and about 900° C.

According to some embodiments, the mineral components may be subjected to at least one heat treatment, followed by co-agglomerating the heat treated mineral components with at least one binder (e.g. silica binder).

### Uses of the Minerals and Mineral Composites

There is further provided herein the various uses of the minerals and mineral composites disclosed herein.

For example, the minerals and mineral composites disclosed herein may be used to absorb organic acid and/or oil and/or water. For example, the minerals and mineral composites disclosed herein may be used to absorb substances that are in solution or suspension with organic acid and/or oil and/or water.

For example, the minerals and mineral composites disclosed herein may be used to absorb organic acid and/or oil and/or water or substances that are in solution or suspension with oil and/or water in a form that is free-flowing. This may be advantageous, for example, for administration of the organic acid and/or oil and/or water substance.

For example, the minerals and mineral composites disclosed herein may be used as a carrier in a functional composition. This means that the mineral or mineral composite absorbs a particular substance to enable it to be administered in a particular form. For example, the mineral or mineral composition may prevent agglomeration or clumping of the functional ingredient (e.g. particular animal feed additive or fertilizer) and may thus make it easier to distribute. For example, the mineral or mineral composite may give the functional ingredient a more solid form and/or prevent the functional ingredient from being washed away from the composition/composite and thus improve the administration or distribution of the functional ingredient.

For example, the minerals and mineral composites disclosed herein may be used as a carrier in an animal feed composition. For example, the minerals and mineral composites disclosed herein may be used as a carrier in a fertilizer composition.

The minerals and mineral composites may, for example, be used as a carrier for one or more additives in animal feed, such as vitamins, organic acids, organic oils, choline chloride solutions, pigment dispersions, antioxidants, antibiotics, molasses, emulsifiers and lubricants.

The present invention thus further relates to the functional compositions disclosed herein, which comprise one or more of the minerals or mineral composites disclosed herein, together with a functional ingredient (e.g. particular animal feed additive or fertilizer). The minerals or mineral composites may be in accordance with any aspect or embodiment of the invention, including all combinations thereof.

Where the minerals and mineral composites disclosed herein are used as carriers, for example in animal feed compositions, the mineral or mineral composite may have an organic acid-absorption capacity of at least about 200 g/100 g of the mineral or mineral composition, for example at least about 250 g/100 g of the mineral or mineral composite, for example at least about 300 g/100 g of the mineral or mineral composite.

The minerals and mineral composites disclosed herein may also be used in various personal care products, for example in deodorant compositions. For example, the minerals and mineral composites disclosed herein may be used to absorb sweat and/or odour. The use of the minerals and mineral compositions disclosed herein in deodorant compositions may, for example, improve the drying time of the composition after it is applied to skin. The use of the minerals and mineral compositions disclosed herein in deodorant compositions may, for example, improve the skin feel of the composition, for example it may give a nude skin effect (skin feels as if there are no residues thereon) and/or it may not provoke or increase skin itching.

Where the minerals and mineral compositions disclosed herein are used in personal care products (e.g. deodorant), the mineral or mineral composite may have an organic acid-absorption capacity equal to or greater than about 220 g/100 g of the mineral or mineral composite (e.g. where the mineral is diatomaceous earth), for example at least about 250 g/100 g of the mineral or mineral composite, for example at least about 270 g/100 g of the mineral or mineral composite (e.g. where the mineral is perlite).

The present invention thus further relates to personal care compositions comprising one or more of the minerals or mineral composites disclosed herein. For example, the present invention relates to deodorant compositions comprising one or more of the minerals or mineral composites disclosed herein. The minerals or mineral composites may be in accordance with any aspect or embodiment of the invention, including all combinations thereof.

### EXAMPLES

### Example 1

The organic acid- and water-absorption capacities of various minerals was determined by the method described above. The oil used was a commercially available blend of organic acids and surfactants, Fylax®. The results are shown in Table 1 below. The base flow energy (BFE) of some of these minerals was also determined by the method described above at 200 g of organic acid (Fylax®) per 100 g of mineral.

The synthetic silica used was Tixosil 38A® (Rhodin), having a particle size of about 60 μm. The diatomaceous earth used had a d₁₀ of 1 μm, a d₅₀ of 9 μm and a d₉₀ of 18 μm. The spray-died diatomaceous earth was made from the same diatomaceous earth that was tested. The spray-dried granules had a d₁₀ of 9 μm, a d₅₀ of 40 μm and a d₉₀ of 71 μm. The granulated diatomaceous earth was made from the same diatomaceous earth that was tested by adding 75 g of a 3.8 wt % PVOH solution to 100 g of the diatomaceous earth and then drying at 110° C. overnight. The granulated diatomaceous earth had a d₁₀ of 4 μm, a d₅₀ of 15 μm and a d₉₀ of 43 μm. The expanded and un-milled perlite had a d₁₀ of about 100 μm, a d₅₀ of about 400 μm and a d₉₀ of about 900 μm. The expanded and classified perlite had a d₁₀ of about 20 μm, a d₅₀ of about 60 μm and a d₉₀ of about 130 μm. The expanded and milled perlite had a d₁₀ of 13 μm, a d₅₀ of 38 μm and a d₉₀ of 50 μm. The calcium silicate had a d₁₀ of about 6 μm, a d₅₀ of about 18 μm and a d₉₀ of about 40 μm. The median equivalent particle diameter (d₅₀ value) and other particle size properties referred to in Examples 1 to 3 are as measured by laser light particle size analysis using a CILAS (Compagnie Industrielle des Lasers) 1064 instrument. The (CILAS) measurements use a particle size measurement as determined by laser light particle size analysis using a CILAS (Compagnie Industrielle des Lasers) 1064 instrument. In this technique, the size of particles in powders, suspensions and emulsions may be measured using the diffraction of a laser beam, based on application of the Fraunhofer theory. The term d₅₀ (CiLAS) used herein is the value determined in this way of the particle diameter at which there are 50% by volume of the particles which have a diameter less than the d₅₀ value. The CILAS 1064 instrument normally provides particle size data to two decimal places.

TABLE 1

| | Organic | | BFE at 200 g |
|---|---|---|---|
| | acid- | Water- | organic |
| | absorption | absorption | acid per |
| | capacity | capacity | 100 g of |
| | (g acid/100 g | (g acid/100 g | mineral |
| Mineral | of mineral) | of mineral) | (mJ) |
| Synthetic Silica | 360 | 355 | 900 |
| Diatomaceous Earth | 300 | 280 | — |
| Spray-Dried | 380 | 355 | 910 |
| Diatomaceous Earth | | | |
| (3% PVOH binder) | | | |
| Granulated | 350 | 355 | — |
| Diatomaceous Earth | | | |
| (with % PVOH) | | | |
| Perlite (Expanded and | 860 | 745 | 370 |
| un-milled) | | | |
| Perlite (Expanded and | 420 | 400 | 360 |
| classified) | | | |
| Perlite (Expanded and | 370 | 355 | — |
| milled) | | | |
| Calcium Silicate | 480 | 460 | — |
| (derived from | | | |
| diatomaceous earth) | | | |

### Example 2

The base flow energy of various minerals containing different levels of water or organic acid (commercially available blend of organic acids and surfactants, Fylax®) was determined by the method described above.

Commercial product 1 is the spray-dried synthetic silica Sipernat 22200, Commercial product 2 is the granulated synthetic silica Tixosil 38A® (Rhodia). Commercial product 3 is the granulated synthetic silica Tixosil 38A®, pre-loaded with the organic acid Fylax®. The spray-dried diatomaceous earth is the mineral described in Example 1 above.

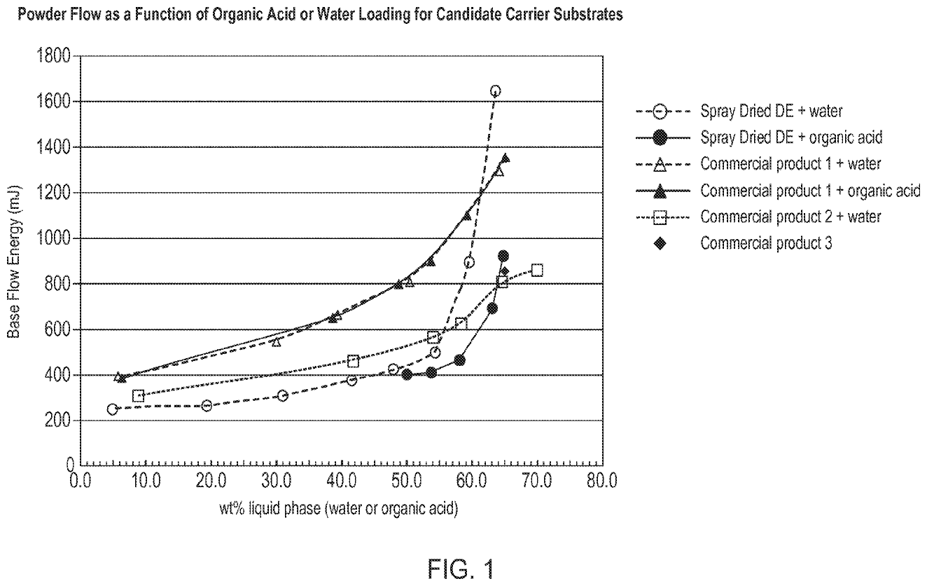

The results are shown in FIG. 1. The x axis of FIG. 1 shows the total amount of liquid (water or organic acid) in the composition. For example, 65 wt % of organic acid means that the composition comprises 65 g of acid for 100−65=35 g of mineral, which is equivalent to 185 g per 100 g of mineral. It was surprisingly found that the spray-dried diatomaceous earth remained free-flowing at relatively high water- and organic acid-loading levels. The performance was similar to that obtained using commercial product 3, which is known to be used in the animal feed industry.

In addition, a different diatomaceous earth having a d₁₀ of 0.6 μm, a d₅₀ of 8.5 μm and a d₉₀ of 18 μm, granulated with 3% polyvinyl alcohol binder, which was prepared in an Eirich mixer and then screened, was found to have a BFE of 1200 mJ when the mineral had a water content of 150 g/100 g of the mineral.

### Example 3

Various minerals were mixed with synthetic sweat and left at 37° C. for 24 hours. Olfactometric analyses were performed to determine odour concentration and to quantify odorant levels. A low odour concentration shows greater odour absorption. Sensory analyses were also conducted to provide information on the sensory properties of the sample products.

For each mineral product, a sample was prepared containing a mixture of 4 g of mineral powder with 6.8 ml of synthetic sweat. The mixture was placed in a Nalophan® bag and after mixing, the closed bag was placed in an oven at 37° C. for 24 hours before olfactometric analysis. In parallel, a bag containing only sweat was prepared in the same manner to serve as a reference.

Odour concentration refers to the persistence of an odour and its resistance to dilution. The higher the odour concentration value, the more difficult it is to dissipate the smell. The dilution factor is determined by a panel of six people. These individuals were selected by Odournet to detect defects in olfactory perception in compliance with EN 13725 standard.

Intensity measurements (based on German standard VDI 3882 part 1) were conducted with a panel of six people selected according to EN 13725 and in relation to a reference scale of n-butanol. During analyses, the individuals smell the product samples directly and assess their intensity on a scale of 0-5 (0=imperceptible, 1=very low, 2=low, 3=medium, 4=high, 5=very high). The relationship between intensity and odour concentration is not linear and is unique to each odorous gas mixture.

The talc had a d₁₀ of about 6 μm, a d₅₀ of about 26 μm and a d₉₀ of about 60 μm. The perlite had a SiO₂ content of 76.0 wt %, an Al₂O₃ content of 12.9 wt %, a K₂O content of 4.6 wt %, a Na₂O content of 3.5 wt % and a d₁₀ of about 8 μm, a d₅₀ of about 25 μm and a d₉₀ of about 50 μm. The diatomaceous earth had a SiO₂ content of 93.0 wt %, an Al₂O₃ content of 2.2 wt %, a Fe₂O₃ content of 1.2 wt % and a d₁₀ of about 3 μm, a d₅₀ of about 12 μm and a d₉₀ of about 45 μm. The particle size distribution of the samples was measured by laser diffraction (CILAS) as described above.

The results are shown in Table 2 below.

TABLE 2

| | Odour Concentration | |
|---|---|---|
| Sample | (UOE/m3) | Intensity |
| Synthetic sweat | 370 | 3.8 |
| Talc | 350 | 2.3 |
| Perlite | 270 | 1.8 |
| Diatomaceous Earth | 190 | 1.8 |

Exemplary deodorant compositions with and without the diatomaceous earth were prepared. It was found that the deodorant composition comprising the diatomaceous earth had a quicker drying time that the deodorant composition that did not comprise the diatomaceous earth. In addition, the deodorant composition comprising diatomaceous earth was found by testers to be easier to spread than the composition without the diatomaceous earth and have a better skin feel (no residues and no unpleasant feeling such as itching).

### Example 4

A commercially available expanded perlite product having a d₅₀ of 20 μm, a specific gravity of 2.1 and a bulking value of 90, and a calcium silicate product derived from diatomaceous earth were used as the feed materials to make high absorption composite materials.

17.5 g of sodium silicate was dispersed in 70 g of Dl water and then slowly added to 350 g of perlite mixture in a Hobart food mixer. The same amount of sodium silicate solution was used for all the perlite mixtures with different ratios. After mixing in for 15 minutes, the mixture was brushed through a 20 mesh screen with a 0.841 mm opening. The oversize particles were broken and forced through the screen by brushing, After drying in a 150° C. oven overnight, the material was brushed through a 30 mesh (0.6 mm opening) screen.

The compositions prepared are shown in Table 3 below.

TABLE 3

| | Perlite | Calcium | Sodium | Water |
|---|---|---|---|---|
| Agglomerate | (g) | Silicate (g) | Silicate (g) | (g) |
| 1 | 87.5 | 262.5 | 17.5 | 70 |
| 2 | 175.0 | 175.0 | 17.5 | 70 |
| 3 | 262.5 | 87.5 | 17.5 | 70 |

The oil-absorption and water-absorption capacities of the perlite and calcium silicate minerals alone and the agglomerates were determined by the method described above using linseed oil. The bulk density of the samples was determined by measuring the weight of a given volume of the loosely packed sample. The wet density of the samples was also determined by placing a sample of known weight from about 1.00 to about 2.00 g in a calibrated 15 ml centrifuge tube to which deionized water is added to make up a volume of approximately 10 ml. The mixture is shaken thoroughly until all of the sample is wetted and no powder remains and additional deionized water is added around the top of the centrifuge tube to rinse down any mixture adhering to the side of the tube from shaking. The tube is centrifuged for 5 minutes at 2500 rpm on an IEC Centra® MP-4R centrifuge, equipped with a Model 221 swinging bucket rotor (International Equipment Company; Needham Heights, Mass., USA). Following centrifugation, the tube is carefully removed without disturbing the solids, and the level (i.e., volume) of the settled matter is measured in cm³. The centrifuged wet density of powder is readily calculated by dividing the sample weight by the measured volume. The particle size distribution of the samples was measured by laser diffraction using a Leeds & Northrup Microtrac Model X-100. The instrument is fully automated, and the results are obtained using a volume distribution formatted in geometric progression of 100 channels, generally running for 30 seconds with the filter on. The distribution is characterized using an algorithm to interpret data from the diffraction pattern which assumes the particles have spherical shape characterized by a diameter, d. For example, a median particle diameter is identified by the instrument as d₅₀, that is, 50% of the total particle volume is accounted for by particles having a diameter equal to or less than this value. The pore volume and pore diameter of the samples were also measured using a mercury porosimeter as described above.

The results are shown in Tables 4 and 5 Belo

TABLE 4

| | | | | Pore | Pore |
|---|---|---|---|---|---|
| | d₁₀ | d₅₀ | d₉₀ | Volume | Diameter |
| Sample | (μm) | (μm) | (μm) | (mL/g) | (μm) |
| Perlite | 9.34 | 21.26 | 46.80 | 4.87 | 3.23 |
| Calcium | 11.58 | 20.04 | 36.91 | 6.11 | 1.44 |
| Silicate | | | | | |
| Agglomerate | 11.26 | 20.20 | 37.84 | 5.94 | 2.02 |
| 1 | | | | | |
| Agglomerate | 10.72 | 20.49 | 40.19 | 5.20 | 2.53 |
| 2 | | | | | |
| Agglomerate | 9.67 | 20.74 | 44.44 | 5.70 | 3.06 |
| 3 | | | | | |

TABLE 5

| | Water- | Oil- | | |
|---|---|---|---|---|
| | absorption | absorption | Bulk | Wet |
| | capacity | capacity | density | density |
| Sample | (%) | (%) | (lb/cf) | (lb/cf) |
| Perlite | 198 | 179 | 5.1 | 16.3 |
| Calcium Silicate | 438 | 392 | 5.5 | 10.1 |
| Agglomerate 1 | 382 | 366 | 6.1 | 11.1 |
| Agglomerate 2 | 378 | 335 | 6.1 | 11.8 |
| Agglomerate 3 | 340 | 300 | 5.9 | 12.6 |

It was surprisingly found that the agglomerates comprising both perlite and calcium silicate minerals had a greater oil-absorption capacity and water-absorption capacity than the mean oil- and water-absorption capacities of the individual minerals.

### Example 5

A diatomaceous earth product having a d₅₀ of 21.26 μm, doe of 46.8 μm, d₁₀ of 9.34 μm, specific gravity of 2.3 and bulking value of 19.2, and a calcium silicate product having a d₅₀ of 19.93 μm, d₉₀ of 36.54 μm, d₁₀ of 11.42 μm and specific gravity of 2.6 were used as the feed materials to make high absorption composite materials. The particle size properties were measured by laser analysis using a Microtrac instrument as described herein.

5 g of a sodium silicate binder was dispersed in 10 g of water and added to DE and calcium silicate in a food mixer. The same amount of binder was used for all the mixtures with different ratios. After mixing for 15 minutes, the mixture was brushed through a 20 mesh screen with a 0,841 mm opening. The oversize particles were broken and forced through the screen by brushing. After drying in a 150° C. oven overnight, the material was brushed through a 30 mesh (0.6 mm) screen.

The compositions prepared are shown in Table 6 below.

TABLE 6

| | DE | Calcium | Binder | Water |
|---|---|---|---|---|
| Composite | (g) | Silicate (g) | (g) | (g) |
| 1 | 75.0 | 25.0 | 5 | 10 |
| 2 | 50,0 | 50.0 | 5 | 10 |
| 3 | 25.0 | 75.0 | 5 | 10 |

The oil-absorption and water-absorption capacities of the DE and calcium silicate minerals alone and the composites were determined by the method described above using linseed oil.

The wet density of the samples was also determined by placing a sample of known weight from about 1.00 to about 2.00 g in a calibrated 15 ml centrifuge tube to which deionized water is added to make up a volume of approximately 10 ml. The mixture is shaken thoroughly until all of the sample is wetted and no powder remains and additional deionized water is added around the top of the centrifuge tube to rinse down any mixture adhering to the side of the tube from shaking. The tube is centrifuged for 5 minutes at 2500 rpm on an IEC Centra® MP-4R centrifuge, equipped with a Model 221 swinging bucket rotor (International Equipment Company; Needham Heights, Mass., USA). Following centrifugation, the tube is carefully removed without disturbing the solids, and the level (i.e., volume) of the settled matter is measured in cm³. The centrifuged wet density of powder is readily calculated by dividing the sample weight by the measured volume.

The surface area of the samples was determined according to the BET method by the quantity of nitrogen adsorbed on the surface of said particles so to as to form a monomolecular layer completely covering said surface (measurement according to the BET method, AFNOR standard X11-621 and 622 or ISO 9277). In certain embodiments, specific surface is determined in accordance with ISO 9277, or any method equivalent thereto.

Colour may be measured by any appropriate measurement technique now known to the skilled artisan or hereafter discovered. In one embodiment, the method for determining the colour of the products of this application uses Hunter scale L, a, b colour data collected on a Spectro/plus Spectrophotometer (Colour and Appearance Technology, Inc., Princeton, N.J.). The L value indicates the level of light or dark, the a-value indicates the level of redness or greenness, and the b-value indicates the level of yellowness or blueness. BLB (blue light brightness) was calculated from the L, a and b value data. A Krypton-filled incandescent lamp is used as the light source. The instrument is calibrated according to the manufacturer's instructions, generally using a highly polished black glass standard and a factory calibrated white opal glass standard. A plastic plate having a depression machined into it is filled with sample, which is then compressed with a smooth-faced plate using a circular pressing motion. The smooth-faced plate is carefully removed to insure an even, unmarred surface. The sample is then placed under the instrument's sample aperture for the measurements.

The results are shown in Table 7.

TABLE 7

| | Oil- | Water- | | | | | | |
|---|---|---|---|---|---|---|---|---|
| | absorption | absorption | Wet | Surface | | | | |
| | capacity | capacity | density | area | | | | |
| Sample | (%) | (%) | (lb/cf) | (m²/g) | L | a | b | BLB |
| DE | 167 | 211 | 16.3 | 2 | 94.75 | 0.09 | 1.82 | 87.31 |
| Calcium Silicate | 409 | 408 | 10.1 | 74 | 83.30 | 0.62 | 3.83 | 64.83 |
| Composite 1 | 233 | 374 | 11.1 | 18 | 89.92 | 0.53 | 3.58 | 76.26 |
| Composite 2 | 289 | 324 | 11.8 | 37 | 86.92 | 0.65 | 3.95 | 70.65 |
| Composite 3 | 350 | 274 | 12.6 | 52 | 84.64 | 0.66 | 3.96 | 66.85 |

It was surprisingly found that the composites had a greater oil-absorption capacity and water-absorption capacity than the oil- and water-absorption capacities of DE alone. In addition, the surface-area of the composites was higher than the surface-area of DE alone. Further, the colour of the composites was better than the colour of the calcium silicate alone.

1-94. (canceled)

95. A silica-based mineral or silicate-based mineral, wherein the silica-based mineral or silicate-based mineral:
a) has an organic acid and/or oil absorption capacity equal to or greater than about 140 g/100 g of the silica-based mineral or silicate-based mineral mineral; and/or
b) is in the form of a free-flowing granulate;
wherein the silica-based mineral or silicate-based mineral is not spray-dried.

96. The silica-based mineral or silicate-based mineral of claim 95, wherein the silica-based mineral or silicate-based mineral has an organic acid and/or oil absorption capacity equal to or greater than about 100 g/100 g of the silica-based mineral or silicate-based mineral.

97. The silica-based mineral or silicate-based mineral of claim 95, wherein the silica-based mineral or silicate-based mineral has a base flow energy (BFE) equal to or less than about 1200 mJ at an organic acid content of 185 g/100 g of the silica-based mineral or silicate-based mineral.

98. The silica-based mineral or silicate-based mineral of claim 95, wherein the silica-based mineral or silicate-based mineral has a BFE equal to or less than about 1000 mJ at an organic acid content of 185 g/100 g of the silica-based mineral or silicate-based mineral.

99. The silica-based mineral or silicate-based mineral of claim 95, wherein the silica-based mineral or silicate-based mineral has a water absorption capacity equal to or greater than about 50 g/100 g of the silica-based mineral or silicate-based mineral.

100. The silica-based mineral or silicate-based mineral of claim 95, wherein the silica-based mineral or silicate-based mineral has a BFE equal to or less than about 1200 mJ at a water content of 150 g/100 g of the silica-based mineral or silicate-based mineral.

101. The silica-based mineral or silicate-based mineral of claim 95, wherein the silica-based mineral or silicate-based mineral is naturally-derived.

102. The silica-based mineral or silicate-based mineral of claim 95, wherein the silica-based mineral or silicate-based mineral is perlite.

103. The silica-based mineral or silicate-based mineral of claim 95, wherein the silica-based mineral or silicate-based mineral is diatomaceous earth.

104. The silica-based mineral or silicate-based mineral of claim 95, wherein the silica-based mineral or silicate-based mineral is calcium silicate that is derived from a natural mineral.

105. The silica-based mineral or silicate-based mineral of claim 95, further comprising a binder.

106. The silica-based mineral or silicate-based mineral of claim 95, further comprising substantially spherical granules.

107. The silica-based mineral or silicate-based mineral of claim 95, further comprising substantially spherical granules, wherein each substantially spherical granule has a mineral shell surrounded by a hollow core.

108. A functional composition comprising a silica-based mineral or silicate-based mineral, wherein the silica-based mineral or silicate-based mineral:
a) has an organic acid and/or oil absorption capacity equal to or greater than about 140 g/100 g of the silica-based mineral or silicate-based mineral; and/or
b) is in the form of a free-flowing granulate;
wherein the silica-based mineral or silicate-based mineral is not spray-dried.

109. The functional composition of claim 108, wherein the functional composition is an animal feed composition or a fertilizer composition.

110. The functional composition of claim 108, wherein the functional composition is a personal care composition.

111. The functional composition of claim 110, wherein the functional composition is a deodorant composition.

112. The functional composition of claim 108, wherein the silica-based mineral or silicate-based mineral is perlite.

113. The functional composition of claim 108, wherein the silica-based mineral or silicate-based mineral is diatomaceous earth.

114. The silica-based mineral or silicate-based mineral of claim 95, wherein the silica-based mineral or silicate-based mineral comprises:
a BET surface area of 5 m²/g to about 80 m²/g;
a median equivalent particle diameter (d₅₀) value of 5 to 30 microns;
a water absorption capacity equal to or greater than about 250 g/100 g of the silica-based mineral or silicate-based mineral; and
an L whiteness equal to or greater than about 80.
